(19) United States
(12) Patent Application Publication
(10) Pub. No.: US 2007/0122845 A1
(43) Pub. Date: May 31, 2007

(54) Methods for identifying GPR83 agonists and GPR83 antagonists capable of modulating regulatory T cell function

(75) Inventors: Tetsu Kawano, Kobe (JP); Junro Kuromitsu, Inashiki-Gun (JP); Akiko Hamaguchi, Kobe (JP); Keiko Yamaguchi, Bethesda, MD (US); Takashi Seiki, Tsukuba-shi (JP); Kazuma Takase, Tsukuba-shi (JP)

Correspondence Address: LAHIVE & COCKFIELD, LLP, ONE POST OFFICE SQUARE, BOSTON, MA, 02109-2127, US

(73) Assignee: Eisai R&D Management Co., Ltd., Tokyo (JP)

(21) Appl. No.: 11/522,025

(22) Filed: Sep. 15, 2006

Related U.S. Application Data
(60) Provisional application No. 60/718,482, filed on Sep. 19, 2005.
(60) Provisional application No. 60/789,477, filed on Apr. 5, 2006.

(51) Int. Cl. C40B 30/06; C40B 40/02; C40B 40/04; A01K 67/027

(52) U.S. Cl. 435/7.1; 800/11; 800/10

(57) ABSTRACT

The present invention provides methods for identifying a GPR83 agonist capable of stimulating a regulatory T cell function and methods for identifying a GPR83 antagonist capable of suppressing a regulatory T cell function.

### RELATED APPLICATIONS

This application claims priority to U.S. Provisional Patent Application Ser. No. 60/718482, filed on Sep. 19, 2005 and U.S. Provisional Application No. 60/789477, filed Apr. 5, 2006, the entire contents of each of which are incorporated herein by this reference.

### BACKGROUND OF THE INVENTION

The immune system provides the human body with a means to recognize and defend itself against microorganisms, viruses, and substances recognized as foreign and potentially harmful. Classical immune responses are initiated when antigen-presenting cells present an antigen to CD4+ T helper (Th) lymphocytes resulting in T cell activation, proliferation, and differentiation of effector T lymphocytes. Following exposure to antigens, such as that which results from infection or the grafting of foreign tissue, naïve T cells differentiate into Th1 and Th2 cells with differing functions. Th1 cells produce interferon gamma (IFN-γ) and interleukin 2 (IL-2) (both associated with cell-mediated immune responses). Th1 cells play a role in immune responses commonly involved in the rejection of foreign tissue grafts as well as many autoimmune diseases. Th2 cells produce cytokines such as interleukin-4 (IL-4), and are associated with antibody-mediated immune responses such as those commonly involved in allergies and allergic inflammatory responses such as allergic rhinitis and asthma. Th2 cells may also contribute to the rejection of foreign grafts. In numerous situations, this immune response is desirable, for example, in defending the body against bacterial or viral infection, inhibiting the proliferation of cancerous cells and the like. However, in other situations, such effector T cells are undesirable, e.g., in a graft recipient.

Whether the immune system is activated by or tolerized to an antigen depends upon the balance between T effector cell activation and T regulatory cell activation. T regulatory cells are responsible for the induction and maintenance of immunological tolerance. These cells are T cells which produce low levels of IL-2, IL-4, IL-5, and IL-12. Regulatory T cells produce TNFα, TGFβ, IFN-γ, and IL-10, albeit at lower levels than effector T cells. Although TGFβ is the predominant cytokine produced by regulatory T cells, the cytokine is produced at lower levels than in Th1 or Th2 cells, e.g., an order of magnitude less than in Th1 or Th2 cells. Regulatory T cells can be found in the CD4+CD25+ population of cells (see, e.g., Waldmann and Cobbold. 2001. Immunity. 14:399). Regulatory T cells actively suppress the proliferation and cytokine production of Th1, Th2, or naïve T cells which have been stimulated in culture with an activating signal (e.g., antigen and antigen presenting cells or with a signal that mimics antigen in the context of MHC, e.g., anti-CD3 antibody, plus anti-CD28 antibody).

Until now, undesirable immune responses have been treated with immunosuppressive drugs, which inhibit the entire immune system, i.e., both desired and undesired immune responses. General immunosuppressants must be administered frequently, for prolonged periods of time, and have numerous harmful side effects. Withdrawal of these drugs generally results in relapse of disease. Thus, there is a need for agents that preferentially modulate the effector or regulatory arm of the immune system without modulating the entire immune system.

### SUMMARY OF THE INVENTION

The present invention is based, at least in part, on the finding that GPR83 (a glucocorticoid-induced receptor first described by Harrigan, M. T. et al. (1991) Mol Endocrin 5:1331-1338) is differentially expressed, both at the mRNA and the protein level, in regulatory T cells (Treg cells). The present invention is also based, at least in part, on the finding that brain derived fractions containing a potential ligand for GPR83 are able to specifically stimulate CD25⁺CD4⁺ regulatory T cells and augment their immunoregulatory activity, e.g., by activating CD25⁺CD4⁺ regulatory T cells to produce cytokines, such as IL-10 and INF-γ.

Accordingly, in one aspect, the present invention provides an assay for identifying a GPR83 agonist capable of stimulating a regulatory T cell function. The method includes contacting a test compound with an indicator composition comprising a GPR83 polypeptide, and determining the ability of the test compound to stimulate the activity of the GPR83 polypeptide, wherein stimulation of the activity of the GPR83 polypeptide indicates that the test compound is capable of stimulating a regulatory T cell function, thereby identifying the test compound as a GPR83 agonist capable of stimulating a regulatory T cell function.

In yet another aspect, the invention provides an assay for identifying a GPR83 agonist capable of stimulating a regulatory T cell function by contacting a test compound with an indicator composition comprising a GPR83 polypeptide, and determining the ability of the test compound to stimulate a regulatory T cell function which is mediated by a GPR83 polypeptide, thereby identifying the test compound as a GPR83 agonist capable of stimulating a regulatory T cell function. The method may further include determining the effect of the test compound on a regulatory T cell function using an in vivo assay. In one embodiment, the in vivo assay may include the use of an animal model for an allergic disease or an autoimmune disease

In one embodiment, the test compound is a member of a library of test compounds and the indicator composition comprising a GPR83 polypeptide is contacted with each member of the library of test compounds. In another embodiment, the test compound is a member of a library of test compounds and wherein the indicator composition comprising a GPR83 polypeptide is contacted with at least half the members of the library of test compounds.

In one embodiment, the indicator composition is a cell expressing a recombinant GPR83 polypeptide. For example, the cell may be engineered to express the GPR83 polypeptide by introducing into the cell an expression vector encoding the GPR83 polypeptide. In yet another embodiment, the indicator composition comprises an indicator cell which contains the GPR83 polypeptide and a reporter gene sensitive to an activity of the GPR83 polypeptide. In one embodiment, the indicator composition is a Foxp3 containing T cell.

In one embodiment, the assays of the invention comprise measuring intracellular adenylyl cyclase activity or intracellular calcium concentration in the presence and in the absence of the test compound and subsequently testing the ability of the test compound to stimulate a regulatory T cell function.

In one embodiment, the regulatory T cell function which is mediated by a GPR83 polypeptide is suppression of the production of an effector cytokine, such as IL-2 or IL-4.

In one embodiment, the regulatory T cell function which is mediated by a GPR83 polypeptide is suppression of the function of an effector T cell, such as a T helper cell, e.g., a Th1 or a Th2 cell, and a cytotoxic T cell (Tc). In another embodiment, the regulatory T cell function which is mediated by a GPR83 polypeptide is suppression of the proliferation of Th1 or Th2 cells. In yet another embodiment, the regulatory T cell function which is mediated by a GPR83 polypeptide is suppression of cytokine production by Th1 or Th2 cells.

In another aspect, the invention provides an assay for identifying a GPR83 antagonist capable of suppressing regulatory T cell function. The method includes contacting a test compound with an indicator composition comprising a GPR83 polypeptide, and determining the ability of the test compound to suppress a regulatory T cell function which is mediated by a GPR83 polypeptide, thereby identifying the test. compound as a GPR83 antagonist capable of suppressing a regulatory T cell function. The method may further comprise determining the effect of the test compound on a T regulatory cell function using an in vivo assay. In one embodiment, the in vivo assay comprises the use of an animal model for HIV or an animal model of a tumor.

In one embodiment, the test compound is a member of a library of test compounds and the indicator composition comprising a GPR83 polypeptide is contacted with each member of the library of test compounds. In another embodiment, the test compound is a member of a library of test compounds and wherein the indicator composition comprising a GPR83 polypeptide is contacted with at least half the members of the library of test compounds.

In one embodiment, the indicator composition is a cell expressing a recombinant GPR83 polypeptide. In one embodiment, the cell has been engineered to express the GPR83 polypeptide by introducing into the cell an expression vector encoding the GPR83 polypeptide. In yet another embodiment, the indicator composition comprises an indicator cell comprising a GPR83 polypeptide and a reporter gene sensitive to an activity of the GPR83 polypeptide. In one embodiment, the indicator composition is a Foxp3 containing T cell.

In one embodiment, the assays of the invention comprise measuring intracellular adenylyl cyclase activity or intracellular calcium concentration in the presence and in the absence of the test compound and subsequently testing the ability of the test compound to suppress a regulatory T cell function. In another embodiment, the regulatory T cell function which is mediated by a GPR83 polypeptide is suppression of the production of an effector cytokine, such as IL-2 or IL-4.

In one embodiment, the regulatory T cell function which is mediated by a GPR83 polypeptide is suppression of the function of an effector T cell, such as a T helper cell, e.g., a Th1 or a Th2 cell, and a cytotoxic T cell (Tc). In another embodiment, the regulatory T cell function which is mediated by a GPR83 polypeptide is suppression of the proliferation of Th1 or Th2 cells. In yet another embodiment, the regulatory T cell function which is mediated by a GPR83 polypeptide is suppression of cytokine production by Th1 or Th2 cells.

Other features and advantages of the invention will be apparent from the following detailed description and claims.

### BRIEF DESCRIPTION OF THE DRAWINGS

FIG. 1 graphically depicts the transcriptome analysis of Foxp3-transduced mouse CD25−CD4+ T cells.

FIG. 2 graphically depicts the results of quantitative real time PCR experiments demonstrating that mGPR83 expression is exclusive to CD25+CD4+ Treg cells.

FIG. 3 graphically depicts the results of a mGPR83 quantification to determine the lymphoid system specificity of mGPR83.

FIG. 4 graphically depicts the results of experiments demonstrating that human GPR83 is also predominantly expressed in CD4+CD25+ human Treg cells.

FIG. 5 graphically depicts the results of experiments demonstrating that human GPR83 is also predominantly expressed in Human Treg cells.

FIG. 6 graphically depicts the tissue distribution of hFOXP3 and hGPR83.

FIG. 7 graphically depicts the results of experiments confirming the specificity of mGPR83 expression on Treg cells at the protein level.

FIG. 8 graphically depicts the results of experiments demonstrating that substantial ligand activity is detected in the mouse brain derived active fraction. The third step of the C18 reverse-phase HPLC (Vydac 218TP54, 4.6 mm×250 mm) elution profile of the crude ligand of GPR83 is depicted. The black bars indicate the specific activities to GPR83 and the white ones the specific activities to GPR37 obtained by a PLAP assay (as described in Example 8).

FIG. 9 graphically depicts the results of experiments designed to analyze Treg function in vitro.

FIG. 10 graphically depicts the results of experiments demonstrating that a mouse brain derived GPR83 ligand specifically stimulates CD25⁺CD4⁺ T cells and augments their immunoregulatory activity.

FIG. 11 graphically depicts the results of experiments demonstrating that a mouse brain derived ligand for GPR83 activates CD25⁺CD4⁺ Treg cells to produce cytokines.

FIG. 12 graphically depicts the results of experiments demonstrating that the mouse brain derived ligand for GPR83 activates the immunoregulatory function of CD25⁺CD4⁺ Treg cells.

### DETAILED DESCRIPTION OF THE INVENTION

In classical immune responses, effector T cell (Teff) responses dominate over responses of regulatory T cells (Tregs) resulting in antigen removal. Tolerance initiates with the same steps as the classical activation pathway (i.e., antigen presentation and T - cell activation), but factors including, but not limited to, the abundance of antigen, the means by which it is presented to the T cell, and the relative availability of CD4+ cell help lead to the proliferation of a distinct class of lymphocytes called regulatory T cells. Just as effector T cells mediate classical immune responses, regulatory T cells mediate tolerogenic responses. However, unwanted or misdirected immune responses, such as those associated with allergy, autoimmune diseases, organ rejection, chronic administration of therapeutic proteins and the like, can lead to conditions in the body which are undesirable and which, in some instances, can prove fatal. The dominance or shifting of balance of regulatory T cells over effector T cells results in antigen preservation and immunological tolerance.

The present invention is based, at least in part, on the finding that GPR83 (a glucocorticoid-induced receptor first described by Harrigan, M. T. et al. (1991) Mol Endocrin 5:1331-1338) is differentially expressed, both at the mRNA and the protein level, in regulatory T cells. The present invention is also based, at least in part, on the finding that brain derived fractions containing a potential ligand for GPR83 are able to specifically stimulate CD25⁺CD4⁺ regulatory T cells and augment their immunoregulatory activity, e.g., by activating CD25⁺CD4⁺ regulatory T cells to produce cytokines, such as IL-10 and INF-γ.

The present invention provides methods for identifying a GPR83 agonist capable of stimulating a regulatory T cell function and methods for identifying a GPR83 antagonist capable of suppressing a regulatory T cell function. The GPR83 agonists identified using the methods described herein are useful for treating a subject having a condition that would benefit from a stimulation of regulatory T cell function, e.g., transplant rejection; allergic diseases and autoimmune diseases. The GPR83 antagonists identified using the methods described herein are useful for treating a subject having a condition that would benefit from a suppression of regulatory T cell function, e.g., a disease associated with viral infections of immune cells (such as AIDS) or cancer.

Before further description of the invention certain terms are, for convenience, described below.

L. Definitions

The term “GPR83”, also known as “glucocorticoid-induced receptor” (“GIR”), “GPR72”, “rp-23”, and “HCEPT09” refers to the orphan G-coupled protein receptor (GPCR or GPR), first identified as a gene induced by glucocorticoids and cAMP by Harrigan, M. T., et al. (1991) Mol Endocrinol. 5(9):1331-8. The nucleic acid sequence and the amino acid sequence of the human GPR83 are known in the art and can be found in GenBank accession number, gi:33354257 (NP057624), the contents of which are incorporated herein in their entirety by reference. The nucleic acid sequence and the amino acid sequence of the murine GPR83 (mGPR83) are also known in the art and can be found in GenBank accession number, gi:193516 (M80481), the contents of which are incorporated herein in their entirety by reference.

As used herein, the term “condition that would benefit from stimulation of regulatory T cell function” includes diseases, disorders, or conditions which would benefit from a stimulation of regulatory T cell function and/or a suppression of effector T cell function. For example, this term includes diseases, disorders, or conditions that would benefit from the suppression of the function of helper T cells (Th), e.g., Th1 and Th2 cells, and/or the function of cytotoxic T cells (Tc). This term also includes diseases, disorders, or conditions that would benefit from the suppression of effector T cell proliferation, and/or the suppression of effector T cell cytokine, e.g., IL-2 or IL-4, production. Non-limiting examples of such diseases, disorders, or conditions, include transplant rejection; atherosclerosis; allergic diseases (e.g., asthma, chronic obstructive pulmonary disease (COPD), eczema, rhinitis, atopic dermatitis and urticaria); and autoimmune diseases (e.g., inflammatory bowel syndrome, type 1 diabetes, rheumatoid arthritis, multiple sclerosis, myasthenia gravis, systemic lupus erythematosis, autoimmune thyroiditis, atopic dermatitis, eczematous dermatitis, psoriasis, Sjögren's Syndrome, alopecia areata, allergic responses due to arthropod bite reactions, Crohn's disease, conjunctivitis, ulcerative colitis, asthma, allergic asthma, cutaneous lupus erythematosus, autoimmune uveitis, idiopathic thrombocytopenia, chronic active hepatitis, lichen planus, Crohn's disease, Juvenile idiopathic arthritis, alopecia universals, autoimmune uveitis, autoimmune hemolytic anemia, pernicious anemia (due to autoimmune gastritis) and chromic autoimmune hepatitis).

Atherosclerosis is described in, for example, Ait-Oufella, H. et al. (2006) Nat Med 12:178-180. Autoimmune inflammatory diseases are described in, for example, Sakaguchi S, et al. (1995) J. Immunol. 155(3):1151-64; Gambineri E, et al. (2003) Curr Opin Rheumatol. 15(4):430-5; and Kriegel M A, et al. (2004) J Exp Med. 199(9):1285-91. Transplantation related diseases are described in, for example, Matsuoka KI, et al. (2005) Blood Epub ahead of print; Hoffmann P, et al. (2005) Curr Top Microbiol Immunol. 293:265-85; Zorn E, et al. (2005) Blood; Ikemoto T, et al. (2004) J Med Invest. 51(3-4):178-85; Taylor P A, et al. (2004) Blood 104(12):3804-12; Edinger M, et al. (2003) Nat Med. 9(9):1144-50; and Guo L, et al. (2003) Transpl Immunol. 12(1):41-8. Allergy, asthma are described in, for example, Hawrylowicz C M, et al. (2005) Nat Rev Immunol. 5(4):271-83; Loser K, et al. (2005) Gene Ther. 12(17):1294-304; and Shi H Z, et al. (2005) Allergy 60(8):986-95. Allergy, eczema are described in, for example, Saint-Mezard P, et al. (2004) Eur J Dermatol. 14(5):284-95; and Gambineri E, et al. 2003) Curr Opin Rheumatol. 15(4):430-5. Allergic rhinitis is described in Francis J N, et al. (2003) J Allergy Clin Immunol. 111(6):1255-61. Atopy, atopic dermaitis is described in, for example, Ling E M, et al. (2004) Lancet. 363(9409):608-15. Urticaria is described in, for example, Nieves D S, et al. (2004) Arch Dermatol. 140(4):466-72. Inflammatory bowel disease is described in, for example, Uhlig H H, et al. (2005) Springer Semin Immunopathol. 27(2): 167-180; Kanai T, et al. (2005) Expert Opin Biol Ther. 5(4):451-62; Coombes J L, et al. (2005) Immunol Rev. 204:184-94; and Mottet C, et al (2003) J Immunol. 170(8):3939-43. Inflammatory bowel disease, Crohn's disease is described in, for example, Makita S, et al. (2004) J Immunol. 173(5):3119-30; and Olson T S, et al. (2004) J Clin Invest. 114(3):389-98. Ulcerative colitis is described in, for example, Weinstock J V, et al. (2005) Springer Semin Immunopathol. 27(2):249-7 1. Type I diabetes is described in, for example, Piccirillo C A, et al. (2005) Ann N Y Acad Sci. 1051:72-87; Ott P A, et al. (2005) Cell Immunol.; and Chatenoud L, et al. (2005) Int Rev Immunol. 24(3-4):247-67. Rheumatoid arthritis is described in, for example, Vigna-Perez M, et al. (2005) Clin Exp Immunol. 141(2):372-80; Morgan M E, et al. (2005) Arthritis Rheum. 52(7):2212-21; Ruprecht C R, et al. (2005) J Exp Med. 201(11):1793-803; Kelchtermans H, et al. (2005) Arthritis Res Ther. 7(2):R402-15; and Frey O, et al. (2005) Arthritis Res Ther. 7(2):R291-301. Juvenile idiopathic arthritis is described in, for example, de Kleer I M, et al. (2004) J Immunol. 172(10):6435-43. Psoriasis is described in, for example, Sugiyama H, et al. (2005) J Immunol. 174(l):164-73; and Bos J D et al. (2005) Br J Dermatol. 152(6):1098-107. Multiple sclerosis is described in, for example, Beyersdorf N, et al. (2005) J Exp Med. 202(3):445-55; Vandenbark A A (2005) Curr Drug Targets Inflamm Allergy. 4(2):217-29; Hong J, et al. (2005) Proc Natl Acad Sci USA. 102(18):6449-54; Viglietta V, et al. (2004) J Exp Med. 199(7):971-9; and McGeachy M J, et al. (2005) J Immunol. 175(5):3025-32. Myasthenia gravis is described in, for example, Luther C, et al. (2005) J Neuroimmunol. 164(1-2):124-8; Ben-David H, et al. (2005) Proc Natl Acad Sci USA. 102(6):2028-33; and Sun Y, et al. (2004) Clin Immunol. 112(3):284-9. Systemic lupus erythematosus is described in, for example, Chen Y, et al. (2005) J Immunol. 175(2):1080-9; and Wolf D, et al. (2005) J Am Soc Nephrol. 16(5): 1360-70. Autoimmune thyroiditis is described in, for example, Gangi E, et al. (2005) J Immunol. 174(11):7006-13; Morris GP, et al. (2005) J Immunol. 174(5):3111-6; and Kriegel M A, et al. (2004) J Exp Med. 199(9):1285-91. Alopecia areata is described in, for example, Zoller M, et al. (2002) J Invest Dermatol. 118(6):983-92. Alopecia universalis is described in, for example, Nieves D S, et al. (2004) Arch Dermatol. 140(4):466-72. Allergic response to arthropod bite is described in, for example, Zuleger C L, et al. (2005) Vaccine 23(24):3181-6. Uveoretinitis, autoimmune uveitis is described in, for example, Takeuchi M, et al. (2004) Invest Ophthalmol Vis Sci. 45(6): 1879-86. Autoimmne hemolytic anemia is described in, for example, Mqadmi A, et al. (2005)Blood 105(9):3746-8; and Nieves D S, et al. (2004) Arch Dermatol. 140(4):466-72. Idiopathic thrombocytopemia is described in, for example, Nieves D S, et al. (2004) Arch Dermatol. 140(4):466-72. Chronic active hepatitis, viral is described in, for example, Furuichi Y, et al. (2005) World J Gastroenterol. 11(24):3772-7; Rushbrook S M, et al. (2005) J Virol. 79(12):7852-9; Stoop J N, et al. (2005) Hepatology 41(4):771-8; and Cabrera R, et al. (2004) Hepatology 40(5):1062-71. Chronic autoimmune hepatitis is described in, for example, Longhi M S, et al. (2005) J Autoimmun. 25(1):63-71; and Longhi MS, et al. (2004) J Hepatol. 41(1l):31-7. Celiac sprue is described in, for example, Popat S, et al. (2002) Ann Hum Genet. 66(Pt 2):125-37. Lichen planus is described in, for example, Hasseus B, et al. (2001) Scand J Immunol. 54(5):516-24.

As used herein the term “agonist” of a GPR83 polypeptide or “GPR83 agonist” is intended to include compounds (e.g., small molecules, peptidic compounds, non-peptidic compounds, e.g., polypeptide analogues, antibodies, or fragments thereof) which stimulate or maintain the activity of the GPR83 polypeptide. For example, a GPR83 agonist can stimulate or retain substantially the same, or a subset, of the biological activities of the naturally occurring form of GPR83. Such GPR83 agonists include molecules which stimulate the expression and/or activity of GPR83 (such as the ability of GPR83 to mediate a regulatory T cell function) or a GPR83 target molecule. Exemplary GPR83 agonists include small molecules, peptidic or non-peptidic molecules (e.g., peptidic or non-peptidic molecules designed based on the peptide isolated in Example 8), and antibodies or fragments thereof (e.g., antibodies such as those generated in Example 7). GPR83 agonists can be identified using the screening assays described herein.

As used herein the term “antagonist” of a GPR83 polypeptide or “GPR83 antagonist” is intended to include compounds (e.g., small molecules, peptidic compounds, non-peptidic compounds, such as polypeptide analogues, antibodies, or fragments thereof) which antagonize the activity of the GPR83 polypeptide. For example, a GPR83 antagonist can inhibit one or more of the activities of the naturally occurring form of GPR83 by, for example, competitively inhibiting a cellular activity of GPR83. Such GPR83 antagonists include molecules which suppress the expression and/or activity of GPR83, such as for example, suppress the ability of GPR83 to mediate a regulatory T cell function.

As used herein, the term “regulatory T cell” or “Treg” includes T cells which are responsible for the induction and maintenance of immunological tolerance. Regulatory T cells produce low levels of IL-2, IL-4, IL-5, and IL-12. Regulatory T cells produce TNFα, TGFβ, IFN-γ, and IL-10, albeit at lower levels than effector T cells. Although TGFβ is the predominant cytokine produced by regulatory T cells, the cytokine is produced at levels less than or equal to that produced by Th1 or Th2 cells, e.g., an order of magnitude less than in Th1 or Th2 cells. Regulatory T cells can be found in the CD4+CD25+ population of cells (see, e.g., Waldmann and Cobbold. 2001. Immunity. 14:399). Regulatory T cells actively suppress the proliferation and cytokine production of Th1, Th2, or naïve T cells which have been stimulated in culture with an activating signal (e.g., antigen and antigen presenting cells or with a signal that mimics antigen in the context of MHC, e.g., anti-CD3 antibody, plus anti-CD28 antibody).

As used herein, the term “regulatory T cell function,” includes an activity exerted by a regulatory T cell, as determined in vivo or in vitro, according to standard techniques. In one embodiment, a regulatory T cell function includes a CD4+CD25+ regulatory T cell function. In another embodiment, a regulatory T cell function includes an IL-10 regulatory T cell function. In yet another embodiment, a regulatory T cell function includes the production of cytokines preferentially associated with regulatory T cells such as, for example, IL-10, TGF-β, or IFN-γ. The term regulatory T cell function includes the initiation and/or maintenance of immunological tolerance. In one embodiment, regulatory T cell function includes the suppression of inflammation. A regulatory T cell function includes the suppression of the activity of effector T cells, e.g., helper T cells (Th), such as, Th1 and Th2 cells and cytotoxic T cells (Tc). For example, a regulatory T cell function includes the suppression of effector T cell or cytotoxic T cell proliferation, and/or the suppression of effector T cell cytokine, e.g., IL-2 or IL-4, production, and/or a biological effect exerted by effector T cells such as, for example, inflammation. In one embodiment, the suppression of effector T cell function is cytokine-dependent. In another embodiment, the suppression of effector T cell function is cytokine-independent.

As used herein, the term “effector T cell” includes cytotoxic T cells (Tc) and helper T cells (Th), e.g., Th1 and Th2 cells. As used herein, the term “effector T cell function” includes an activity exerted by an effector T cell, as determined in vivo or in vitro, according to standard techniques. In one embodiment, an effector T cell function includes the elimination of an antigen by, for example, the production of cytokines preferentially associated with effector T cells, which modulate the activation of other cells, or by cytotoxic activity. In one embodiment, a T effector cell function is a cytotoxic (or cytolytic) T cell (Tc or CTL) function, such as, for example, cytolysis of cells infected with microbes. In another embodiment, a T effector cell function is a Th1 cell function, e.g., mediation of delayed type hypersensitivity responses and macrophage activation. In yet another embodiment, a T effector cell function is a Th2 cell function, e.g., help to B cells (Mosmann and Coffinan, 1989, Annu. Rev. Immunol. 7, 145-173; Paul and Seder, 1994, Cell 76, 241-251; Arthur and Mason, 1986, J Exp. Med. 163, 774-786; Paliard et al., 1988, J Immunol. 141, 849-855; Finkelman et al., 1988, J. Immunol. 141, 2335-2341). In another embodiment, an effector T cell function includes an inflammatory response. In one embodiment, effector T cell function includes the suppression of immunological tolerance. In yet another embodiment, an effector T cell function includes the suppression of the activity of regulatory T cells. For example, an effector T cell function includes the suppression of regulatory T cell proliferation, and/or the suppression of regulatory T cell cytokine, e.g., IL-10 and/or IFN-γ, production, and/or a biological effect exerted by regulatory T cells such as, for example, immunological tolerance. In one embodiment, the suppression of regulatory T cell function is cytokine-dependent. In another embodiment, the suppression of regulatory T cell function is cytokine-independent.

As used herein, the term “immune response” includes immune cell-mediated (e.g., T cell and/or B cell-mediated) immune responses that are influenced by modulation of immune cell activation. Exemplary immune responses include B cell responses (e.g., antibody production, e.g., IgA production), T cell responses (e.g., proliferation, cytokine production and cellular cytotoxicity), and activation of cytokine responsive cells, e.g., macrophages. In one embodiment of the invention, an immune response is T cell mediated. In another embodiment of the invention, an immune response is B cell mediated. As used herein, the term “downregulation” or “suppression” with reference to the immune response includes a diminution in any one or more immune responses, preferably T cell responses, while the term “upregulation” or “stimulation” with reference to the immune response includes an increase in any one or more immune responses, preferably T cell responses. It will be understood that upregulation of one type of immune response may lead to a corresponding downregulation in another type of immune response. For example, upregulation of the production of certain cytokines (e.g., IL-10) can lead to downregulation of cellular immune responses and vice versa. Similarly, upregulation of regulatory T cell function can lead to the downregulation of effector T cell function and vice versa.

As used herein, the term “T helper type 1 response” (Th1 response) refers to a response that is characterized by the production of one or more cytokines selected from IFN-γ, IL-2, TNF, and lymphotoxin (LT) and other cytokines produced preferentially or exclusively by Th1 cells rather than by Th2 cells.

As used herein, a “T helper type 2 response” (Th2 response) refers to a response by CD4⁺ T cells that is characterized by the production of one or more cytokines selected from IL-4, IL-5, IL-6 and IL-10, and that is associated with efficient B cell “help” provided by the Th2 cells (e.g., enhanced IgG1 and/or IgE production).

As used herein, the term “Th1-associated cytokine” is intended to refer to a cytokine that is produced preferentially or exclusively by Th1 cells rather than by Th2 cells. Examples of Th1-associated cytokines include IFN-γ, IL-2, TNF, and lymphtoxin (LT).

As used herein, the term “Th2-associated cytokine” is intended to refer to a cytokine that is produced preferentially or exclusively by Th2 cells rather than by Th1 cells. Examples of Th1-associated cytokines include IL-4, IL-5, and IL-10.

As used herein, the term “treating” includes the application or administration of a GPR83 agonist or a GPR83 antagonist to a subject, or application or administration of a GPR83 agonist or a GPR83 antagonist to an isolated tissue or cell line from a subject, who has a disease, disorder, or condition, a symptom of disease, disorder, or condition, or a predisposition toward a disease, disorder, or condition, with the purpose of curing, healing, alleviating, relieving, altering, remedying, ameliorating, improving or affecting the disease or disorder, at least one symptom of disease disorder, or condition.

As used herein, the term “effective amount” or “therapeutically active amount” refers to the amount of a GPR83 agonist or a GPR83 antagonist that is therapeutically effective, at dosages and for periods of time necessary to achieve the desired result. For example, an effective amount of a GPR83 agonist or a GPR83 antagonist may vary according to factors such as the disease state, age, sex, and weight of the individual, and the ability of agent to elicit a desired response in the subject. Dosage regimens can be adjusted to provide the optimum therapeutic response. For example, several divided doses can be administered daily or the dose can be proportionally reduced as indicated by the exigencies of the therapeutic situation.

As used herein, the term “immune cell” includes cells that are of a hematopoietic origin and that play a role in the immune response. Immune cells include lymphocytes, such as B cells and T cells; natural killer cells; and myeloid cells, such as monocytes, macrophages, eosinophils, mast cells, basophils, and granulocytes.

As used herein, the term “T cell” (i.e., T lymphocyte) is intended to include all cells within the T cell lineage, including thymocytes, immature T cells, mature T cells and the like, from a mammal (e.g., human). Preferably, T cells are mature T cells that express either CD4+ or CD8+, but not both, and a T cell receptor. The various T cell populations described herein can be defined based on their cytokine profiles and their function.

As used herein “progenitor T cells” (“Thp”) are pluripotent cells that express both CD4 and CD8.

As used herein, the term “naïve T cells” includes T cells that have not been exposed to cognate antigen and so are not activated or memory cells. Naïve T cells are not cycling and human naïve T cells are CD45RA+. If naïve T-cells recognize antigen and receive additional signals depending upon but not limited to the amount of antigen, route of administration and timing of administration, they may proliferate and differentiate into various subsets of T cells, e.g., effector T cells.

As used herein, the term “peripheral T cells” refers to mature single positive T cells that leave the thymus and enter the peripheral circulation.

As used herein, the term “differentiated” refers to T cells that have been contacted with a stimulating agent and includes effector T cells (e.g., Th1, Th2) and memory T cells. Differentiated T cells differ in expression of several surface proteins compared to naïve T cells and secrete cytokines that activate other cells.

As used herein, the term “memory T cell” includes lymphocytes which, after exposure to antigen, become functionally quiescent and which are capable of surviving for long periods in the absence of antigen. Human memory T cells are CD45RA-.

The term “small molecule” is a term well-known in the art and includes molecules that are less than about 1000 molecular weight, less than about 800, less than about 750 molecular weight, less than about 700 molecular weight, less than about 650 molecular weight, less than about 600 molecular weight, less than about 550 molecular weight, less than about 500 molecular weight, less than about 450 molecular weight, less than about 400 molecular weight, less than about 350 molecular weight, less than about 350 molecular weight, less than about 250 molecular weight, or less than about 200 molecular weight. In one embodiment, small molecules do not exclusively comprise peptide bonds. In another embodiment, small molecules are not oligomeric. Exemplary small molecule compounds which can be screened for activity according to the methods of the present invention include, but are not limited to, amino acids, peptides, peptidomimetics, carbohydrates, lipids, small organic molecules (e.g., polyketides) (Cane et al. 1998. Science 282:63), natural product extract libraries, or other organic (carbon containing) molecules. Organic small molecules typically have multiple carbon-carbon bonds. In one embodiment, the compounds are small, organic non-peptidic compounds. In another embodiment, a small molecule is not biosynthetic.

As used herein, the term “indicator composition” refers to a composition that includes the GPR83 polypeptide and is suitable for use in the screening assays described herein. For example, an indicator composition can be a cell that naturally expresses GPR83, a cell that has been engineered to express GPR83 by introducing an expression vector encoding GPR83 into the cell, a cell free composition that contains GPR83, an animal, e.g., a transgenic mouse, comprising GPR83, or a cell or tissue derived from such an animal.

As used herein, the term “contacting” a test compound with an indicator composition comprising GPR83 polypeptide is intended to include incubating the test compound and the indicator composition together in vitro (e.g., adding the test compound to cells in culture), or in vivo (e.g., administering the test compound to an animal model of a disease, disorder, or condition). The term “contacting” does not include exposure of cells to a GPR83 agonist that may occur naturally in a subject (i.e., exposure that may occur as a result of a natural physiological process).

As used herein, the term “subject” is intended to include living organisms in which an immune response can be elicited. Preferred subjects are mammals. Particularly preferred subjects are humans. Other examples of subjects include monkeys, dogs, cats, mice, rats cows, horses, goats, sheep as well as other farm and companion animals. Stimulation of regulatory T cell function, in humans as well as veterinary applications, provides a means to regulate disorders arising from aberrant regulatory T cell function in various disease states and is encompassed by the present invention.

II. Screening Methods

The invention further provides methods for identifying a GPR83 agonist (e.g., a peptidic compound, a small molecule, a non-peptidic compound, or an antibody or fragment thereof) that is capable of stimulating regulatory T cell function, e.g., capable of stimulating GPR83 mediated CD4+CD25+ regulatory T cell function, such as suppressing effector T cell function as described herein.

For example, in one embodiment, compounds which stimulate regulatory T cell function by, for example, stimulating the expression and/or activity of GPR83 and/or stimulating a regulatory T cell function mediated by GPR83, and/or stimulating the interaction, e.g., binding, of GPR83 to a target molecule, can be identified using the screening assays described herein.

The ability of a compound to stimulate regulatory T cell function can be determined by, for example, measuring the proliferation of T cells, e.g., regulatory T, e.g., CD4+CD25+, cells, and/or effector T cells, such as cytotoxic T cells and helper T cells, e.g., Th1 and Th2 cells, or by measuring cytokines produced by these cells, e.g., the production Th1-specific and/or Th2-specific cytokines, e.g., IL-2 or IL-4. Additionally, the ability of a compound to modulate regulatory T cell function can be determined by, for example, measuring the expression and/or activity of GPR83. For example, GPR83 is a G-coupled protein receptor and has the ability to stimulate intracellular cAMP or intracellular calcium production as taught in the Examples. Thus, intracellular adenylate cyclase activity, intracellular cAMP concentration, or intracellular calcium concentration may be measured as part of the screening assays described herein. Adenylate cyclase activity is measured, for example, by enzyme immunoassay utilizing commercially available kits from, for example, Stratagene, Inc., La Jolla, Calif. Cytokine production, can be measured, for example, by flow cytometry (see, McNerlan, S E, et al.(2002) Exp Gerontol 37(2-3):227-34) and/or commercially available ELISA assays. The ability of a compound to directly modulate, e.g., increase or stabilize, or decrease or destabilize, the formation of a complex between GPR83 and a binding partner may also be measured.

The screening assays discussed herein can be performed in the presence or absence of other agents. For example, the assays can be performed in the presence of various agents that modulate the activation state of the cell being screened. For example, in one embodiment, agents that transduce signals via the T cell receptor are included. Exemplary activating agents are known in the art and include, but are not limited to, e.g., mitogens (e.g., phytohemagglutinin or concanavalin A), antibodies that react with the T cell receptor or CD3 (in some cases combined with antigen presenting cells or antibodies that react with CD28), or antigen plus antigen presenting cells. In another embodiment, a cytokine or an antibody to a cytokine receptor is included.

In another aspect, the invention pertains to a combination of two or more of the assays described herein. For example, a GPR83 agonist can be identified using a cell-based or a cell-free assay, and the ability of the GPR83 agonist to stimulate regulatory T cell function can be confirmed in vivo, e.g., in an animal such as an animal model for multiple sclerosis (EAE), rheumatoid arthritis, COPD, or allergy.

Moreover, a GPR83 agonist identified as described herein (e.g., a small molecule, a peptidic compound, a polypeptide analog, or an antibody, or fragment thereof) can be used in an animal model to determine the efficacy, toxicity, or side effects of treatment with such an agent. Alternatively, a GPR83 agonist identified as described herein can be used in an animal model to determine the mechanism of action of such an agent. For example, an agent can be tested in art recognized animal models of human diseases (e.g., EAE as a model of multiple sclerosis and NOD mice as a model for diabetes) or other well characterized animal models of human autoimmune diseases. Such animal models include the mrl/lpr/lpr mouse as a model for lupus erythematosus, murine collagen-induced arthritis as a model for rheumatoid arthritis, and murine experimental myasthenia gravis (see Paul ed., Fundamental Immunology, Raven Press, New York, 1989, pp. 840-856). A GPR83 agonist identified as described herein can be administered to test animals and the course of the disease in the test animals can then be monitored using standard methods for the particular model being used. Effectiveness of the GPR83 agonist is evidenced by amelioration of the disease condition in animals treated with the GPR83 agonist as compared to untreated animals (or animals treated with a control agent).

In an embodiment of a screening assay of the invention, once a test compound is identified as a GPR83 agonist, the effect of the test compound can be assayed for an ability to stimulate T regulatory cell function and can be confirmed as a suitable compound for use in the therapeutic methods of the invention, for example, based on measurements of the effects in immune cells, either in vitro (e.g., using cell lines or cells derived from a subject) or in vivo (e.g., using an animal model). Accordingly, the screening methods of the invention can further comprise determining the effect of the GPR83 agonist on at least one T regulatory activity to thereby confirm that a compound has the desired effect.

In one embodiment, the ability of a test compound is further assayed for the ability to modulate an activity associated with a T effector cell, e.g., proliferation and/or cytokine production. In a further embodiment, the ability of a test compound is assayed for the ability to modulate an activity associated with a T regulatory cell, e.g., tolerance. For example, determining the ability of a test compound to modulate tolerance can be determined by subsequent attempts at stimulation of T cells with antigen presenting cells. If the T cells are unresponsive to the subsequent activation attempts, as determined by, for example, IL-2 synthesis and T cell proliferation, a state of tolerance has been induced, e.g., regulatory T cell function has been activated, and alternatively, if IL-2 synthesis is stimulated and T cells proliferate, effector T cell function has been activated. See, e.g., Gimmi, C. D. et al. (1993) Proc. Natl. Acad. Sci. USA 90, 6586-6590; and Schwartz (1990) Science, 248, 1349-1356, for exemplary assay systems that can used as the basis for an assay in accordance with the present invention. Other methods for measuring the diminished activity of tolerized T cells include, without limitation, measuring intracellular calcium mobilization, measuring protein levels of members of the MAP kinase cascade, and/or by measuring the activity of the AP-1 complex of transcription factors in a T cell upon engagement of its T cell receptors. T cell proliferation can be measured, for example, by assaying [³H] thymidine incorporation and measuring protein levels according to methods commonly employed by one of skill in the art. Cytokine levels can be assayed by any number of commercially available kits for immunoassays, including but not limited to, Stratagene, Inc., La Jolla, Calif.

Compounds identified using the assays described herein are useful for treating disorders associated with aberrant regulatory T cell function and/or aberrant GPR83 expression and/or activity, such as those diseases, disorders, or conditions described above in Section II.

The invention further provides methods for identifying a GPR83 antagonist (e.g., a peptidic compound, a small molecule, a non-peptidic compound, or an antibody or fragment thereof) that is capable of suppressing regulatory T cell function, e.g., capable of suppressing GPR83 mediated CD4+CD25+ regulatory T cell function, such as stimulating effector T cell function as described herein. In these methods, all the assays described herein with respect to the identification of a GPR83 agonist may be used, except the opposite effect would be tested. For example, compounds which suppress regulatory T cell function may be identified by detecting a decreased proliferation of regulatory T cells, and/or by detecting an increased proliferation of effector T cells, and/or by detecting an increased production of Th1-specific and/or Th2-specific cytokines, e.g., IL-2 or IL-4.

The screening assays of the invention as well as the test compounds employed therein are described in more detail below.

A. Cell Based Assays

The indicator composition used in the screening assays of the invention can be a cell that expresses a GPR83 polypeptide (and/or one or more other polypeptides or genes, such as a target of GPR83 polypeptides or the Foxp3 gene which is believed to be the “master regulator gene” regulating the expression of various genes in Tregs). For example, a cell that naturally expresses endogenous GPR83 or, more preferably, a cell that has been engineered to express an exogenous GPR83 polypeptide by introducing into the cell an expression vector encoding the polypeptide may be used.

An indicator cell can be transfected with a GPR83 expression vector, incubated in the presence and in the absence of a test compound, and the effect of the compound on the expression of the molecule or on a biological response regulated by GPR83 can be determined. The biological activities of GPR83 include activities determined in vivo, or in vitro, according to standard techniques. A GPR83 activity can be a direct activity, such as an association of GPR83 with a GPR83-target molecule or stimulation of regulatory T cell function. Alternatively, a GPR83 activity is a downstream activity, such as a cellular signaling activity occurring downstream of the interaction of the GPR83 polypeptide with a GPR83 target molecule or a biological effect occurring as a result of the signaling cascade triggered by that interaction. For example, biological activities of GPR83 that may be tested as described herein include: stimulation of regulatory T cell function, the initiation and/or maintenance of immunological tolerance, the suppression of effector T cell, e.g., helper T cell (Th), e.g., Th1 and Th2 cell, and cytotoxic T cell (Tc), function, e.g., the suppression of effector T cell proliferation, the suppression of effector T cell cytokine, e.g., IL-2, production, and/or the stimulation of Foxp3 expression.

To determine whether a test compound modulates GPR83 expression, in vitro transcriptional assays can be performed. To perform such an assay, the full length promoter and enhancer of GPR83 can be operably linked to a reporter gene such as chloramphenicol acetyltransferase (CAT) or luciferase and introduced into host cells.

As used interchangeably herein, the terms “operably linked” and “operatively linked” are intended to mean that the nucleotide sequence is linked to a regulatory sequence in a manner which allows expression of the nucleotide sequence in a host cell (or by a cell extract). Regulatory sequences are art-recognized and can be selected to direct expression of the desired polypeptide in an appropriate host cell. The term regulatory sequence is intended to include promoters, enhancers, polyadenylation signals and other expression control elements. Such regulatory sequences are known to those skilled in the art and are described in Goeddel, Gene Expression Technology: Methods in Enzymology 185, Academic Press, San Diego, Calif. (1990). It should be understood that the design of the expression vector may depend on such factors as the choice of the host cell to be transfected and/or the type and/or amount of polypeptide desired to be expressed.

A variety of reporter genes are known in the art and are suitable for use in the screening assays of the invention. Examples of suitable reporter genes include those which encode chloramphenicol acetyltransferase, beta-galactosidase, alkaline phosphatase or luciferase. Standard methods for measuring the activity of these gene products are known in the art and described herein in the Examples section.

In one embodiment, the level of expression of the reporter gene in the indicator cell in the presence of the test compound is higher than the level of expression of the reporter gene in the indicator cell in the absence of the test compound and the test compound is identified as a compound that stimulates the expression and/or activity of GPR83, and/or regulatory T cell function. In another embodiment, the level of expression of the reporter gene in the indicator cell in the presence of the test compound is lower than the level of expression of the reporter gene in the indicator cell in the absence of the test compound and the test compound is identified as a compound that inhibits the expression and/or activity of GPR83, and/or regulatory T cell function.

A variety of cell types are suitable for use as indicator cells in the screening assay. Preferably a cell line is used which does not normally express GPR83, such as an effector T cell clone, e.g., a Th2 cell clone, or a cell from a GPR83 transgenic animal, such as those described in U.S.2002/0184657 and WO02/03793, the contents of each of which are hereby expressly incorporated herein by reference. As described in U.S.2002/0184657 and WO02/03793, cells overexpressing GPR83 have been produced and show expression in brain, pharynx, testis and prostate. Thus, cells from these organs, or cell lines generated from those cells can be used in the screening assays described herein. Non-lymphoid cell lines can also be used as indicator cells, such as the HEK293 cell line described in the examples below.

Cells for use in the subject assays include both eukaryotic and prokaryotic cells. For example, in one embodiment, a cell is a bacterial cell. In another embodiment, a cell is a fungal cell, such as a yeast cell. In another embodiment, a cell is a vertebrate cell, e.g., an avian cell or a mammalian cell (e.g., a murine cell, or a human cell). In a preferred embodiment, however, the cell is a mammalian cell, such as a human or murine cell.

The ability of a test compound to stimulate a GPR83 mediated regulatory T cell function may also be determined by determining the ability of the test compound to modulate GPR83 binding to a target molecule. Determining the ability of the test compound to modulate GPR83 binding to a target molecule (e.g., an intracellular binding partner) can be accomplished, for example, by coupling the GPR83 target molecule with a radioisotope, enzymatic or fluorescent label such that binding of the GPR83 target molecule to GPR83 can be determined by detecting the labeled GPR83 target molecule in a complex. Alternatively, GPR83 could be coupled with a radioisotope, enzymatic or fluorescent label such that binding of the compound to GPR83 can be determined by detecting the labeled GPR83 compound in a complex. For example, GPR83 targets can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a test compound to interact with GPR83 without the labeling of any of the interactants. For example, a microphysiometer can be used to detect the interaction of a compound with GPR83 without the labeling of either the compound or the GPR83 (McConnell, H. M. et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and GPR83.

In another embodiment, a different (i.e., non-GPR83) molecule acting in a pathway involving GPR83 that acts upstream or downstream of GPR83 can be included in an indicator composition for use in a screening assay. Compounds identified in a screening assay employing such a molecule would also be useful in modulating GPR83 activity, albeit indirectly.

In yet another aspect of the invention, the GPR83 polypeptide or fragments thereof can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other polypeptides, which bind to or interact with GPR83 (“GPR83-binding proteins”) and are involved in GPR83 activity. Such GPR83-binding proteins are also likely to be involved in the propagation of signals by the GPR83 polypeptides or GPR83 targets as, for example, downstream elements of a GPR83-mediated signaling pathway. Alternatively, such GPR83-binding polypeptides are likely to be modulators of GPR83 activity.

The two-hybrid system is based on the modular nature of most transcription factors, which consist of separable DNA-binding and activation domains. Briefly, the assay utilizes two different DNA constructs. In one construct, the gene that codes for a GPR83 polypeptide is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait and the “prey” proteins are able to interact, in vivo, forming a GPR83-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the polypeptide which interacts with the GPR83 polypeptide.

B. Cell-free Assays

In one embodiment the indicator composition used in the screening assays of the invention is a cell-free composition that includes GPR83 and/or one or more non-GPR83 polypeptides. GPR83 or a non-GPR83 polypeptide which acts upstream or downstream of GPR83 in a pathway involving GPR83 expressed by recombinant methods in a host cells or culture medium can be isolated from the host cells, or cell culture medium using standard methods for purifying polypeptides, for example, by ion-exchange chromatography, gel filtration chromatography, ultrafiltration, electrophoresis, and immunoaffinity purification with antibodies specific for GPR83 to produce protein that can be used in a cell free composition. Alternatively, an extract of GPR83 or non-GPR83 expressing cells can be prepared for use as cell-free composition.

In one embodiment, compounds that specifically stimulate regulatory T cell function by stimulating GPR83 activity are identified based on their ability to stimulate the interaction of GPR83 with a target molecule to which GPR83 binds. Suitable assays are known in the art that allow for the detection of protein-protein interactions (e.g., immunoprecipitations, fluorescent polarization or energy transfer, two-hybrid assays and the like). By performing such assays in the presence and absence of test compounds, these assays can be used to identify compounds that stimulate the interaction of GPR83 with a target molecule and, thus, stimulate regulatory T cell function.

In one embodiment, the amount of binding of GPR83 to the target molecule in the presence of the test compound is greater than the amount of binding of GPR83 to the target molecule in the absence of the test compound, in which case the test compound is identified as a compound that enhances or stabilizes binding of GPR83 and/or stimulates regulatory T cell function. In another embodiment, the amount of binding of the GPR83 to the target molecule in the presence of the test compound is less than the amount of binding of the GPR83 to the target molecule in the absence of the test compound, in which case the test compound is identified as a compound that inhibits or destabilizes binding of GPR83 and/or inhibits regulatory T cell function.

Binding of the test compound to the GPR83 polypeptide can be determined either directly or indirectly as described above. Determining the ability of the GPR83 polypeptide to bind to a test compound can also be accomplished using a technology such as real-time Biomolecular Interaction Analysis (BIA) (Sjolander, S. and Urbaniczky, C. (1991) Anal. Chem. 63:2338-2345; Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In the methods of the invention for identifying test compounds that modulate an interaction between GPR83 polypeptide and a target molecule, the full-length GPR83 polypeptide may be used in the method, or, alternatively, only portions of the GPR83 may be used. The degree of interaction between GPR83 polypeptides and the target molecule can be determined, for example, by labeling one of the polypeptides with a detectable substance (e.g., a radiolabel), isolating the non-labeled polypeptide and quantitating the amount of detectable substance that has become associated with the non-labeled polypeptide. The assay can be used to identify test compounds that either stimulate or inhibit the interaction between the GPR83 protein and a target molecule. A test compound that stimulates the interaction between the GPR83 polypeptide and a target molecule is identified based upon its ability to increase the degree of interaction between the GPR83 polypeptide and a target molecule as compared to the degree of interaction in the absence of the test compound. A test compound that inhibits the interaction between the GPR83 polypeptide and a target molecule is identified based upon its ability to decrease the degree of interaction between the GPR83 polypeptide and a target molecule as compared to the degree of interaction in the absence of the compound.

In more than one embodiment of the above assay methods of the present invention, it may be desirable to immobilize either GPR83 or a GPR83 target molecule, to facilitate separation of complexed from uncomplexed forms of one or both of the polypeptides, or to accommodate automation of the assay. Binding of a test compound to a GPR83 polypeptide, or interaction of a GPR83 polypeptide with a GPR83 target molecule in the presence and absence of a test compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the polypeptides to be bound to a matrix. For example, glutathione-S-transferase/GPR83 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target polypeptide or GPR83 polypeptide, and the mixture incubated under conditions conducive to complex formation (e.g., at physiological conditions for salt and pH). Following incubation, the beads or microtitre plate wells are washed to remove any unbound components, the matrix is immobilized in the case of beads, and complex formation is determined either directly or indirectly, for example, as described above. Alternatively, the complexes can be dissociated from the matrix, and the level of GPR83 binding or activity determined using standard techniques.

Other techniques for immobilizing polypeptides on matrices can also be used in the screening assays of the invention. For example, either a GPR83 polypeptide or a GPR83 target molecule can be immobilized utilizing conjugation of biotin and streptavidin. Biotinylated GPR83 polypeptide or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies which are reactive with GPR83 polypeptide or target molecules but which do not interfere with binding of the GPR83 polypeptide to its target molecule can be derivatized to the wells of the plate, and unbound target or GPR83 polypeptide is trapped in the wells by antibody conjugation. Methods for detecting such complexes, in addition to those described above for the GST-immobilized complexes, include immunodetection of complexes using antibodies reactive with the GPR83 polypeptide or target molecule, as well as enzyme-linked assays which rely on detecting an enzymatic activity associated with the GPR83 polypeptide or target molecule.

C. Test Compounds

A variety of test compounds can be evaluated using the screening assays described herein. In certain embodiments, the compounds to be tested can be derived from libraries (i.e., are members of a library of compounds). While the use of libraries of peptides is well established in the art, new techniques have been developed which have allowed the production of mixtures of other compounds, such as benzodiazepines (Bunin et al. (1992). J Am. Chem. Soc. 114:10987; DeWitt et al. (1993). Proc. Natl. Acad. Sci. USA 90:6909) peptoids (Zuckermann. (1994). J. Med. Chem. 37:2678) oligocarbamates (Cho et al. (1993). Science. 261:1303- ), and hydantoins (DeWitt et al. supra). An approach for the synthesis of molecular libraries of small organic molecules with a diversity of 10⁴-10⁵ as been described (Carell et al. (1994). Angew. Chem. Int. Ed. Engl. 33:2059; Carell et al. (1994) Angew. Chem. Int. Ed. Engl. 33:2061).

The test compounds of the present invention can be obtained using any of the numerous approaches in combinatorial library methods known in the art, including: biological libraries; spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Compound Des. 12:145). Other exemplary methods for the synthesis of molecular libraries can be found in the art, for example in: Erb et al.(1994). Proc. Natl. Acad. Sci. USA 91:11422; Horwell et al. (1996) Immunopharmacology 33:68-; and in Gallop et al. (1994); J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. USA. 87:6378-6382); (Felici (1991) J. Mol. Biol. 222:301-310). In still another embodiment, the combinatorial polypeptides are produced from a cDNA library.

Exemplary compounds which can be screened for activity include, but are not limited to, peptides, nucleic acids, carbohydrates, small molecules, and natural product extract libraries.

Candidate/test compounds include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)2, Fab expression library fragments, and epitope-binding fragments of antibodies); 4) small organic and inorganic molecules (e.g., molecules obtained from combinatorial and natural product libraries); 5) enzymes (e.g., endoribonucleases, hydrolases, nucleases, proteases, synthatases, isomerases, polymerases, kinases, phosphatases, oxido-reductases and ATPases), and 6) mutant forms or GPR83 molecules, e.g., dominant negative mutant forms of the molecules.

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Compounds identified in the subject screening assays can be used in methods of stimulating regulatory T cell function. It will be understood that it may be desirable to formulate such compound(s) as pharmaceutical compositions (described supra) prior to contacting them with cells.

Once a test compound is identified that directly or indirectly stimulates regulatory T cell function, by one of the variety of methods described hereinbefore, the selected test compound (or “compound of interest”) can then be further evaluated for its effect on cells, for example by contacting the compound of interest with cells either in vivo (e.g., by administering the compound of interest to a subject) or ex vivo (e.g., by isolating cells from the subject and contacting the isolated cells with the compound of interest or, alternatively, by contacting the compound of interest with a cell line) and determining the effect of the compound of interest on the cells, as compared to an appropriate control (such as untreated cells or cells treated with a control compound, or carrier, that does not modulate the biological response). Compounds of interest can also be identified using structure based drug design using techniques known in the art.

III. Stimulatory or Inhibitory Agents

According to the methods of the invention, GPR83 agonists or GPR83 antagonists are identified. Examples of GPR83 agonists or GPR83 antagonists include small molecules, peptidic compounds, non-peptidic compounds (such as polypeptide analogues), antibodies, or fragments thereof, and are described in further detail below.

The term “peptides” or “peptidic compounds,” as used herein, is intended to include molecules comprised only of natural amino acid residues (i.e., alanine, arginine, aspartic acid, asparagine, cysteine, glutamic acid, glutamine, glycine, histidine, isoleucine, leucine, lysine, methionine, phenylalanine, proline, serine, threonine, tryptophan, tyrosine and valine) linked by peptide bonds, or other residues whose structures can be determined by standard sequencing methodologies (e.g., direct sequencing of the amino acids making up the peptides or sequencing of nucleic acid molecules encoding the peptide). The term “peptide” or “peptidic compound” is not intended to include molecules structurally related to peptides, such as peptide derivatives, peptide analogues or peptidomimetics, whose structures cannot be determined by standard sequencing methodologies but rather must be determined by more complex chemical strategies, such as mass spectrometric methods.

The term “non-peptide compounds”, as used herein, is intended to include compounds comprising at least one molecule other than a natural amino acid residue, wherein the structures of the compounds cannot be determined by standard sequencing methodologies but rather must be determined by more complex chemical strategies, such as mass spectrometric methods. Preferred non-peptide compounds are those that, although not composed entirely of natural amino acid residues, are nevertheless related structurally to peptides, such as peptidomimetics, peptide derivatives and peptide analogues. As used herein, a “derivative” of a compound X (e.g., a peptide) refers to a form of X in which one or more reactive groups on the compound have been derivatized with a substituent group. Examples of peptide derivatives include peptides in which an amino acid side chain, the peptide backbone, or the amino- or carboxy-terminus has been derivatized (e.g., peptidic compounds with methylated amide linkages). As used herein an “analogue” of a compound X refers to a compound which retains chemical structures of X necessary for functional activity of X yet which also contains certain chemical structures which differ from X. An example of an analogue of a naturally-occurring peptide is a peptide which includes one or more non-naturally-occurring amino acids. As used herein, a “mimetic” of a compound X refers to a compound in which chemical structures of X necessary for functional activity of X have been replaced with other chemical structures which mimic the conformation of X. Examples of peptidomimetics include peptidic compounds in which the peptide backbone is substituted with one or more benzodiazepine molecules (see e.g., James, G. L. et al. (1993) Science 260:1937-1942) and “retro-inverso” peptides (see U.S. Pat. No. 4,522,752 by Sisto), described further below.

The term mimetic, and in particular, peptidomimetic, is intended to include isosteres. The term “isostere” as used herein is intended to include a chemical structure that can be substituted for a second chemical structure because the steric conformation of the first structure fits a binding site specific for the second structure. The term specifically includes peptide back-bone modifications (i.e., amide bond mimetics) well known to those skilled in the art. Such modifications include modifications of the amide nitrogen, the α-carbon, amide carbonyl, complete replacement of the amide bond, extensions, deletions or backbone crosslinks. Several peptide backbone modifications are known, including ψ[CH₂S], ψ[CH₂NH], ψ[CSNH₂], ψ[NHCO], ψ[COCH₂], and ψ[(E) or (Z) CH═CH]. In the nomenclature used above, ψ indicates the absence of an amide bond. The structure that replaces the amide group is specified within the brackets. Other examples of isosteres include peptides substituted with one or more benzodiazepine molecules (see e.g., James, G. L. et al. (1993) Science 260:1937-1942), peptoids (R. J. Simon et al. (1992) Proc. Natl. Acad. Sci. USA 89:9367-9371), and the like.

Other possible modifications of peptides include an N-alkyl (or aryl) substitution (ψ[CONR]), backbone crosslinking to construct lactams and other cyclic structures, or retro-inverso amino acid incorporation (ψ[NHCO]). By “inverso” is meant replacing L-amino acids of a sequence with D-amino acids, and by “retro-inverso” or “enantio-retro” is meant reversing the sequence of the amino acids (“retro”) and replacing the L-amino acids with D-amino acids. For example, if the parent peptide is Thr-Ala-Tyr, the retro modified form is Tyr-Ala-Thr, the inverso form is thr-ala-tyr, and the retro-inverso form is tyr-ala-thr (lower case letters refer to D-amino acids). Compared to the parent peptide, a retro-inverso peptide has a reversed backbone while retaining substantially the original spatial conformation of the side chains, resulting in a retro-inverso isomer with a topology that closely resembles the parent peptide. See Goodman et al. “Perspectives in Peptide Chemistry” pp. 283-294 (1981). See also U.S. Pat. No. 4,522,752 by Sisto for further description of “retro-inverso” peptides.

A GPR83 agonist or a GPR83 antagonist may also be a biologically active portion of GPR83 (i.e., a bioactive fragment of GPR83), or a biologically active portion of a GPR83 ligand.

Bioactive fragments of GPR83 or bioactive fragments of a GPR83 ligand include polypeptides comprising amino acid sequences sufficiently identical to or derived from the amino acid sequence of the subject polypeptide which include less amino acids than the full length protein, and exhibit at least one biological activity of the full-length protein. Typically, biologically active portions comprise a domain or motif with at least one activity of the full-length protein. A biologically active portion of a polypeptide of the invention can be a polypeptide which is, for example, 10, 20, 30, 40, 50, 100, 150, 200, 250, 300, 350, 400, 450, 500, or more amino acids in length. Moreover, other biologically active portions, in which other regions of the protein are deleted, can be prepared by recombinant techniques and evaluated for one or more of the functional activities of a native protein. Mutants can also be utilized as assay reagents, for example, mutants having reduced, enhanced or otherwise altered biological properties identified according to one of the activity assays described herein.

Variants of the GPR83 polypeptide molecule or variants of a ligand for GPR83 which retain biological activity may also be used as GPR83 agonists or antagonists in the methods of the invention. In one embodiment, such a variant polypeptide has at least about 80%, 85%, 90%, 95%, 98% identity to the polypeptide sequence of GPR83.

A GPR3 agonist or a GPR83 antagonist of the present invention can also be an antibody, or a fragment thereof. The term “antibody” as used herein refers to immunoglobulin molecules and immunologically active portions of immunoglobulin molecules, i.e., molecules that contain an antigen binding site which specifically binds (immunoreacts with) an antigen. Examples of immunologically active portions of immunoglobulin molecules include F(ab) and F(ab′)2 fragments which can be generated by treating the antibody with an enzyme such as pepsin as well as VH and VL domains that can be cloned from antibody molecules and used to generate modified antigen binding molecules, such as minibodies or diabodies.

The antibodies of the invention can be used in formulating various therapeutic compositions of the invention or, preferably, provide complementarity determining regions for the production of humanized or chimeric antibodies (described in detail below). The production of non-human monoclonal antibodies, e.g., murine, guinea pig, primate, rabbit or rat, can be accomplished by, for example, immunizing the animal with the antigen of interest, e.g., GPR83 or a fragment thereof (such as that described in Example 7), or with a nucleic acid molecule encoding the antigen of interest, e.g., GPR83. A longer polypeptide comprising GPR83 or an immunogenic fragment of GPR83 or anti-idiotypic antibody of GPR83 can also be used. (see, for example, Harlow & Lane, supra, incorporated by reference for all purposes). Such an immunogen can be obtained from a natural source, by peptide synthesis or by recombinant expression. Optionally, the immunogen can be administered, fused or otherwise complexed with a carrier protein, as described below. Optionally, the immunogen can be administered with an adjuvant.

The term “adjuvant” refers to a compound that when administered in conjunction with an antigen augments the immune response to the antigen, but when administered alone does not generate an immune response to the antigen. Adjuvants can augment an immune response by several mechanisms including lymphocyte recruitment, stimulation of B and/or T cells, and stimulation of macrophages. Several types of adjuvant can be used as described below. Complete Freund's adjuvant followed by incomplete adjuvant is preferred for immunization of laboratory animals.

Rabbits or guinea pigs are typically used for making polyclonal antibodies. Exemplary preparation of polyclonal antibodies, e.g., for passive protection, can be performed as follows. Animals are immunized with 100 μg GPR83, plus adjuvant, and euthanized at 4-5 months. Blood is collected and IgG is separated from other blood components. Antibodies specific for the immunogen may be partially purified by affinity chromatography. An average of about 0.5-1.0 mg of immunogen-specific antibody is obtained per animal, giving a total of 60-120 mg.

Mice are typically used for making monoclonal antibodies. Monoclonals can be prepared against a fragment by injecting the fragment or longer form of GPR83 into a mouse, preparing hybridomas and screening the hybridomas for an antibody that specifically binds to GPR83. Optionally, antibodies are screened for binding to a specific region or desired fragment of GPR83 without binding to other nonoverlapping fragments of GPR83. The latter screening can be accomplished by determining binding of n antibody to a collection of deletion mutants of a GPR83 peptide and determining which deletion mutants bind to the antibody. Binding can be assessed, for example, by Western blot or ELISA. The smallest fragment to show specific binding to the antibody defines the epitope of the antibody. Alternatively, epitope specificity can be determined by a competition assay in which a test and reference antibody compete for binding to GPR83. If the test and reference antibody compete, then they bind to the same epitope (or epitopes sufficiently proximal) such that binding of one antibody interferes with binding of the other. The preferred isotype for such antibodies is mouse isotype IgG2a or equivalent isotype in other species. Mouse isotype IgG2a is the equivalent of human isotype IgG1.

Any of the many well known protocols used for fusing lymphocytes and immortalized cell lines can be applied for the purpose of generating a monoclonal antibody (see, e.g., G. Galfre et. al. . (1977) Nature 266:55052; Gefter et. al. . Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of a protein kinase C theta pathway are detected by screening the hybridoma culture supernatants for antibodies that bind to the antigen, e.g., using a standard ELISA assay.

Alternative to preparing monoclonal antibody-secreting hybridomas, a monoclonal antibody can be identified and isolated by screening a recombinant combinatorial immunoglobulin library (e.g., an antibody phage display library) with an antigen to thereby isolate immunoglobulin library members that bind the antigen. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et. al.. U.S. Pat. No. 5,223,409; Kang et. al.. PCT International Publication No. WO 92/18619; Dower et. al. PCT International Publication No. WO 91/17271; Winter et. al.. PCT International Publication WO 92/20791; Markland et. al.. PCT International Publication No. WO 92/15679; Breitling et. al.. PCT International Publication WO 93/01288; McCafferty et. al.. PCT International Publication No. WO 92/01047; Garrard et. al.. PCT International Publication No. WO 92/09690; Ladner et. al. PCT International Publication No. WO 90/02809; Fuchs et. al.. (1991) BioTechnology 9:1370-1372; Hay et. al.. (1992) Hum; Antibod. Hybridomas 3:81-85; Huse et. al.. (1989) Science 246:1275-1281; Griffiths et. al. (1993) EMBO J 12:725-734; Hawkins et. al. (1992) J. Mol. Biol. 226:889-896; Clarkson et al. (1991) Nature 352:624-628; Gram et al. (1992) PNAS 89:3576-3580; Garrad et al. (1991) BioTechnology 9:1373-1377; Hoogenboom et al. (1991) Nuc. Acid Res. 19:4133-4137; Barbas et. al.. (1991) PNAS 88:7978-7982; and McCafferty et al. Nature (1990) 348:552-554.

The GPR83 agonists or antagonists of the present invention can also be (and preferably are) chimeric and/or humanized antibodies (e.g., chimeric and/or humanized immunoglobulins) specific for GPR83 or a GPR83 ligand. Chimeric and/or humanized antibodies have the same or similar binding specificity and affinity as a mouse or other nonhuman antibodies that provide the starting material for construction of a chimeric or humanized antibody.

A chimeric antibody is one whose light and heavy chain genes have been constructed, typically by genetic engineering, from immunoglobulin gene segments belonging to different species. For example, the variable (V) segments of the genes from a mouse monoclonal antibody may be joined to human constant (C) segments, such as IgG1 and IgG4. Human isotype IgG1 is preferred. A typical chimeric antibody is thus a hybrid protein consisting of the V or antigen-binding domain from a mouse antibody and the C or effector domain from a human antibody.

The term “humanized binding molecule” refers to a binding molecule comprising at least one chain comprising variable region framework residues derived from a human binding molecule chain (referred to as the acceptor immunoglobulin or binding molecule) and at least one complementarity determining region derived from a mouse-binding molecule, (referred to as the donor immunoglobulin or binding molecule). Humanized binding molecules can be produced using recombinant DNA technology, which is discussed below. See for example, e.g., Hwang, W. Y. K., et al. (2005) Methods 36:35; Queen et al., Proc. Natl. Acad. Sci. USA, (1989), 86:10029-10033; Jones et al., Nature, (1986), 321:522-25; Riechmann et al., Nature, (1988), 332:323-27; Verhoeyen et al., Science, (1988), 239:1534-36; Orlandi et al., Proc. Natl. Acad. Sci. USA, (1989), 86:3833-37; U.S. Pat. Nos. US 5,225,539; 5,530,101; 5,585,089; 5,693,761; 5,693,762; 6,180,370, Selick et al., WO 90/07861, and Winter, U.S. Pat. No. 5,225,539 (incorporated by reference in their entirety for all purposes). The constant region(s), if present, are preferably also derived from a human immunoglobulin.

The substitution of mouse CDRs into a human variable domain framework is most likely to result in retention of their correct spatial orientation if the human variable domain framework adopts the same or similar conformation to the mouse variable framework from which the CDRs originated. This is achieved by obtaining the human variable domains from human antibodies whose framework sequences exhibit a high degree of sequence identity with the murine variable framework domains from which the CDRs were derived. The heavy and light chain variable framework regions can be derived from the same or different human antibody sequences. The human antibody sequences can be the sequences of naturally occurring human antibodies or can be consensus sequences of several human antibodies. See Kettleborough et al., Protein Engineering 4:773 (1991); Kolbinger et al., Protein Engineering 6:971 (1993) and Carter et al., WO 92/22653.

Having identified the complementarity determining regions of the murine donor immunoglobulin and appropriate human acceptor immunoglobulins, the next step is to determine which, if any, residues from these components should be substituted to optimize the properties of the resulting humanized antibody. In general, substitution of human amino acid residues with murine should be minimized, because introduction of murine residues increases the risk of the antibody eliciting a human-anti-mouse-antibody (HAMA) response in humans. Art-recognized methods of determining immune response can be performed to monitor a HAMA response in a particular patient or during clinical trials. Patients administered humanized antibodies can be given an immunogenicity assessment at the beginning and throughout the administration of said therapy. The HAMA response is measured, for example, by detecting antibodies to the humanized therapeutic reagent, in serum samples from the patient using a method known to one in the art, including surface plasmon resonance technology (BIACORE) and/or solid-phase ELISA analysis.

Certain amino acids from the human variable region framework residues are selected for substitution based on their possible influence on CDR conformation and/or binding to antigen. The unnatural juxtaposition of murine CDR regions with human variable framework region can result in unnatural conformational restraints, which, unless corrected by substitution of certain amino acid residues, lead to loss of binding affinity.

The selection of amino acid residues for substitution can be determined, in part, by computer modeling. In general, molecular models are produced starting from solved structures for immunoglobulin chains or domains thereof. The chains to be modeled are compared for amino acid sequence similarity with chains or domains of solved three-dimensional structures, and the chains or domains showing the greatest sequence similarity is/are selected as starting points for construction of the molecular model. Chains or domains sharing at least 50% sequence identity are selected for modeling, and preferably those sharing at least 60%, 70%, 80%, 90% sequence identity or more are selected for modeling. The solved starting structures are modified to allow for differences between the actual amino acids in the immunoglobulin chains or domains being modeled, and those in the starting structure. The modified structures are then assembled into a composite immunoglobulin. Finally, the model is refined by energy minimization and by verifying that all atoms are within appropriate distances from one another and that bond lengths and angles are within chemically acceptable limits.

The selection of amino acid residues for substitution can also be determined, in part, by examination of the characteristics of the amino acids at particular locations, or empirical observation of the effects of substitution or mutagenesis of particular amino acids. For example, when an amino acid differs between a murine variable region framework residue and a selected human variable region framework residue, the human framework amino acid should usually be substituted by the equivalent framework amino acid from the mouse antibody when it is reasonably expected that the amino acid: (1) noncovalently binds antigen directly, (2) is adjacent to a CDR region, (3) otherwise interacts with a CDR region (e.g., is within about 3-6 Å of a CDR region as determined by computer modeling), or (4) participates in the VL-VH interface.

Residues which “noncovalently bind antigen directly” include amino acids in positions in framework regions which are have a good probability of directly interacting with amino acids on the antigen according to established chemical forces, for example, by hydrogen bonding, Van der Waals forces, hydrophobic interactions, and the like.

Residues which are “adjacent to a CDR region” include amino acid residues in positions immediately adjacent to one or more of the CDRs in the primary sequence of the humanized immunoglobulin chain, for example, in positions immediately adjacent to a CDR as defined by Kabat, or a CDR as defined by Chothia (See e.g., Chothia and Lesk JMB 196:901 (1987)). These amino acids are particularly likely to interact with the amino acids in the CDRs and, if chosen from the acceptor, may distort the donor CDRs and reduce affinity. Moreover, the adjacent amino acids may interact directly with the antigen (Amit et al., Science, 233:747 (1986), which is incorporated herein by reference) and selecting these amino acids from the donor may be desirable to keep all the antigen contacts that provide affinity in the original antibody.

Residues that “otherwise interact with a CDR region” include those that are determined by secondary structural analysis to be in a spatial orientation sufficient to effect a CDR region. In one embodiment, residues that “otherwise interact with a CDR region” are identified by analyzing a three-dimensional model of the donor immunoglobulin (e.g., a computer-generated model). A three-dimensional model, typically of the original donor antibody, shows that certain amino acids outside of the CDRs are close to the CDRs and have a good probability of interacting with amino acids in the CDRs by hydrogen bonding, Van der Waals forces, hydrophobic interactions, etc. At those amino acid positions, the donor immunoglobulin amino acid rather than the acceptor immunoglobulin amino acid may be selected. Amino acids according to this criterion will generally have a side chain atom within about 3 Å of some atom in the CDRs and must contain an atom that could interact with the CDR atoms according to established chemical forces, such as those listed above.

In the case of atoms that may form a hydrogen bond, the 3 Å is measured between their nuclei, but for atoms that do not form a bond, the 3 Å is measured between their Van der Waals surfaces. Hence, in the latter case, the nuclei must be within about 6 Å (3 Å plus the sum of the Van der Waals radii) for the atoms to be considered capable of interacting. In many cases the nuclei will be from 4 or 5 to 6 Å apart. In determining whether an amino acid can interact with the CDRs, it is preferred not to consider the last 8 amino acids of heavy chain CDR as part of the CDRs, because from the viewpoint of structure, these 8 amino acids behave more as part of the framework.

Amino acids that are capable of interacting with amino acids in the CDRs, may be identified in yet another way. The solvent accessible surface area of each framework amino acid is calculated in two ways: (1) in the intact antibody, and (2) in a hypothetical molecule consisting of the antibody with its CDRs removed. A significant difference between these numbers of about 10 square angstroms or more shows that access of the framework amino acid to solvent is at least partly blocked by the CDRs, and therefore that the amino acid is making contact with the CDRs. Solvent accessible surface area of an amino acid may be calculated based on a three-dimensional model of an antibody, using algorithms known in the art (e.g., Connolly, J. Appl. Cryst. 16:548 (1983) and Lee and Richards, J. Mol. Biol. 55:379 (1971), both of which are incorporated herein by reference). Framework amino acids may also occasionally interact with the CDRs indirectly, by affecting the conformation of another framework amino acid that in turn contacts the CDRs.

The amino acids at several positions in the framework are known to be capable of interacting with the CDRs in many antibodies (Chothia and Lesk, supra, Chothia et al., supra and Tramontano et al., J. Mol. Biol. 215:175 (1990), all of which are incorporated herein by reference). Notably, the amino acids at positions 2, 48, 64 and 71 of the light chain and 26-30, 71 and 94 of the heavy chain (numbering according to Kabat) are known to be capable of interacting with the CDRs in many antibodies. The amino acids at positions 35 in the light chain and 93 and 103 in the heavy chain are also likely to interact with the CDRs. At all these numbered positions, choice of the donor amino acid rather than the acceptor amino acid (when they differ) to be in the humanized immunoglobulin is preferred. On the other hand, certain residues capable of interacting with the CDR region, such as the first 5 amino acids of the light chain, may sometimes be chosen from the acceptor immunoglobulin without loss of affinity in the humanized antibody.

Residues which “participate in the VL-VH interface” or “packing residues” include those residues at the interface between VL and VH as defined, for example, by Novotny and Haber (Proc. Natl. Acad. Sci. USA, 82:4592-66 (1985)) or Chothia et al, supra. Generally, unusual packing residues should be retained in the humanized antibody if they differ from those in the human frameworks.

In general, one or more of the amino acids fulfilling the above criteria is substituted. In some embodiments, all or most of the amino acids fulfilling the above criteria are substituted. Occasionally, there is some ambiguity about whether a particular amino acid meets the above criteria, and alternative variant antibodies are produced, one of which has that particular substitution, the other of which does not. Alternative variant antibodies so produced can be tested in any of the assays described herein for the desired activity, and the preferred antibody selected.

Usually the CDR regions in humanized antibodies are substantially identical, and more usually, identical to the corresponding CDR regions of the donor antibody. Although not usually desirable, it is sometimes possible to make one or more conservative amino acid substitutions of CDR residues without appreciably affecting the binding affinity of the resulting humanized antibody. By conservative substitutions it is meant combinations such as Gly, Ala; Val, Ile, Leu; Asp, Glu; Asn, Gln; Ser, Thr; Lys, Arg; and Phe, Tyr.

Additional candidates for substitution are acceptor human framework amino acids that are unusual or “rare” for a human immunoglobulin at that position. These amino acids can be substituted with amino acids from the equivalent position of the mouse donor antibody or from the equivalent positions of more typical human immunoglobulins. For example, substitution may be desirable when the amino acid in a human framework region of the acceptor immunoglobulin is rare for that position and the corresponding amino acid in the donor immunoglobulin is common for that position in human immunoglobulin sequences; or when the amino acid in the acceptor immunoglobulin is rare for that position and the corresponding amino acid in the donor immunoglobulin is also rare, relative to other human sequences. These criterion help ensure that an atypical amino acid in the human framework does not disrupt the antibody structure. Moreover, by replacing an unusual human acceptor amino acid with an amino acid from the donor antibody that happens to be typical for human antibodies, the humanized antibody may be made less immunogenic.

The term “rare”, as used herein, indicates an amino acid occurring at that position in less than about 20% but usually less than about 10% of sequences in a representative sample of sequences, and the term “common”, as used herein, indicates an amino acid occurring in more than about 25% but usually more than about 50% of sequences in a representative sample. For example, all human light and heavy chain variable region sequences are respectively grouped into “subgroups” of sequences that are especially homologous to each other and have the same amino acids at certain critical positions (Kabat et al., supra). When deciding whether an amino acid in a human acceptor sequence is “rare” or “common” among human sequences, it will often be preferable to consider only those human sequences in the same subgroup as the acceptor sequence.

Additional candidates for substitution are acceptor human framework amino acids that would be identified as part of a CDR region under the alternative definition proposed by Chothia et al., supra. Additional candidates for substitution are acceptor human framework amino acids that would be identified as part of a CDR region under the AbM and/or contact definitions. Notably, CDR1 in the variable heavy chain is defined as including residues 26-32.

Additional candidates for substitution are acceptor framework residues that correspond to a rare or unusual donor framework residue. Rare or unusual donor framework residues are those that are rare or unusual (as defined herein) for murine antibodies at that position. For murine antibodies, the subgroup can be determined according to Kabat and residue positions identified which differ from the consensus. These donor specific differences may point to somatic mutations in the murine sequence which enhances activity. Unusual residues that are predicted to affect binding are retained, whereas residues predicted to be unimportant for binding can be substituted.

Additional candidates for substitution are non-germline residues occurring in an acceptor framework region. For example, when an acceptor antibody chain (i.e., a human antibody chain sharing significant sequence identity with the donor antibody chain) is aligned to a germline antibody chain (likewise sharing significant sequence identity with the donor chain), residues not matching between acceptor chain framework and the germline chain framework can be substituted with corresponding residues from the germline sequence.

In one embodiment, a CDR homology based method is used for humanization (see, e.g., Hwang, W. Y. K., et al. (2005) Methods 36:35, the contents of which are incorporated in their entirety herein by this reference). This method generally involves substitution of mouse CDRs into a human variable domain framework based on similarly structured mouse and human CDRs rather than similarly structured mouse and human frameworks. The similarity of the mouse and human CDRs is generally determined by identifying human genes of the same chain type (light or heavy) that have the same combination of canonical CDR structures as the mouse binding molecules and thus retain three-dimensional conformation of CDR peptide backbones. Secondly, for each of the candidate variable genes with matching canonical structures, residue to residue homology between the mouse and candidate human CDRs is evaluated. Finally, to generate a humanized binding molecule, CDR residues of the chosen human candidate CDR not already identical to the mouse CDR are converted to the mouse sequence. In one embodiment, no mutations of the human framework are introduced into the humanized binding molecule.

Other than the specific amino acid substitutions discussed above, the framework regions of humanized antibodies are usually substantially identical, and more usually, identical to the framework regions of the human antibodies from which they were derived. Of course, many of the amino acids in the framework region make little or no direct contribution to the specificity or affinity of a antibody. Thus, many individual conservative substitutions of framework residues can be tolerated without appreciable change of the specificity or affinity of the resulting humanized antibody. Thus, in one embodiment the variable framework region of the humanized antibody shares at least 85% sequence identity to a human variable framework region sequence or consensus of such sequences. In another embodiment, the variable framework region of the humanized antibody shares at least 90%, preferably 95%, more preferably 96%, 97%, 98% or 99% sequence identity to a human variable framework region sequence or consensus of such sequences. In general, however, such substitutions are undesirable.

The humanized antibodies preferably exhibit a specific binding affinity for antigen of at least 10⁷, 10⁸, 10⁹ or 10¹⁰ M⁻¹. Usually the upper limit of binding affinity of the humanized antibodies for antigen is within a factor of three, four or five of that of the donor immunoglobulin. Often the lower limit of binding affinity is also within a factor of three, four or five of that of donor immunoglobulin. Alternatively, the binding affinity can be compared to that of a humanized antibody having no substitutions (e.g., a antibody having donor CDRs and acceptor FRs, but no FR substitutions). In such instances, the binding of the optimized antibody (with substitutions) is preferably at least two- to three-fold greater, or three- to four-fold greater, than that of the unsubstituted antibody. For making comparisons, activity of the various antibodies can be determined, for example, by BIACORE (i.e., surface plasmon resonance using unlabelled reagents) or competitive binding assays.

Having conceptually selected the CDR and framework components of humanized antibodies, a variety of methods are available for producing such antibodies. Because of the degeneracy of the code, a variety of nucleic acid sequences will encode each antibody amino acid sequence. The desired nucleic acid sequences can be produced by de novo solid-phase DNA synthesis or by PCR mutagenesis of an earlier prepared variant of the desired polynucleotide.

Oligonucleotide-mediated mutagenesis is a preferred method for preparing substitution, deletion and insertion variants of target polypeptide DNA. See Adelman et al. (DNA 2:183 (1983)). Briefly, the target polypeptide DNA is altered by hybridizing an oligonucleotide encoding the desired mutation to a single-stranded DNA template. After hybridization, a DNA polymerase is used to synthesize an entire second complementary strand of the template that incorporates the oligonucleotide primer, and encodes the selected alteration in the target polypeptide DNA.

The variable segments of antibodies produced as described supra (e.g., the heavy and light chain variable regions of chimeric, humanized, or human antibodies) are typically linked to at least a portion of an immunoglobulin constant region (Fc), typically that of a human immunoglobulin. Human constant region DNA sequences can be isolated in accordance with well known procedures from a variety of human cells, but preferably immortalized B cells (see Kabat et al., supra, and Liu et al., W087/02671) (each of which is incorporated by reference in its entirety for all purposes). Ordinarily, the antibody will contain both light chain and heavy chain constant regions. The heavy chain constant region usually includes CH1, hinge, CH2, CH3, and CH4 regions. The antibodies described herein include antibodies having all types of constant regions, including IgM, IgG, IgD, IgA and IgE, and any isotype, including IgG1, IgG2, IgG3 and IgG4. The choice of constant region depends, in part, or whether antibody-dependent complement and/or cellular mediated toxicity is desired. For example, isotopes IgG1 and IgG3 have complement activity and isotypes IgG2 and IgG4 do not. When it is desired that the antibody (e.g., humanized antibody) exhibit cytotoxic activity, the constant domain is usually a complement fixing constant domain and the class is typically IgG1. When such cytotoxic activity is not desirable, the constant domain may be, e.g., of the IgG2 class. Choice of isotype can also affect passage of antibody into e brain. Human isotype IgG1 is preferred. Light chain constant regions can be lambda or kappa. The humanized antibody may comprise sequences from more than one class or isotype. Antibodies can be expressed as tetramers containing two light and two heavy chains, as separate heavy chains, light chains, as Fab, Fab′ F(ab′)2, and Fv, or as single chain antibodies in which heavy and light chain variable domains are linked through a spacer.

Other GPR83 agonists or antagonists that can be used in the methods of the invention are chemical compounds, such as the small molecules. Such compounds can be identified using screening assays that select for such compounds, as described in detail above.

IV. Pharmaceutical Compositions

GPR83 agonists or antagonists identified using the methods of the present invention, can be incorporated into pharmaceutical compositions suitable for administration to a subject. Such compositions typically comprise the agent and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, intramuscular, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fuisidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, GPR83 agonists or antagonists are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations should be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

The pharmaceutical compositions can be included in a container, pack, or dispenser together with instructions for administration.

This invention is further illustrated by the following examples, which should not be construed as limiting. The contents of all references, patents and published patent applications cited throughout this application, as well as the Figures, are incorporated herein by reference.

### EXAMPLES

### Example 1

### Transcriptome Analysis of Foxp3-transduced Mouse CD25−CD4+ T Cells

Aim of Experiment

Foxp3 gene is essential for the development and function of CD25+CD4+ regulatory T cells (Hori S and Sakaguchi S. Science 229: 1057-1061, 2003). In order to identify genes that are regulated by Foxp3 transcriptional factor, mouse Foxp3 was retrovirally transduced in mouse CD25−CD4+ non-regulatory T cells and the transcriptome of the cells was analyzed with normalized representational differential analysis and gene chip analysis.

Materials and Methods

Foxp3-transduced and Empty Vector-transduced T Cells

The open reading frame (ORF) of the Mouse Foxp3 gene (Accession# NM_{—}054039) was amplified by PCR with cDNAs from RNA samples of mouse CD25+CD4+ T cells and the following primer set, 5′-CGGAATTCCGCACCATGCCCAACCCTAGGCCAG-3′ (as forward primer) and 5′-CCGCTCGAGCGGTCAAGGGCAGGGATTGGAGC-3′ (as reverse primer). The resulting DNA fragment was sequenced and subcloned into the EcoRI and XhoI restriction endonuclease sites of the pMX-IRES-EGFP retroviral vector (pMX-Foxp3-IRES-EGFP) (Nosaka T et al, EMBO-J 18: 4754-4765, 1999). The resulting pMX-mFoxp3-IRES-EGFP clone was transfected into BD EcoPack2-293 cell line (BD Biosciences Clontech), by the use of Lipofectamine 2000 reagent (Invitrogen Life Technologies, CA, USA) according to the manufacturer's protocol and the culture supernatant containing ecotropic virus particles was harvested after 48 hours of transfection. Supernatant was applied to mouse CD25−CD4+ T cells freshly isolated from female BALB/c spleen (Charles River, Mass., USA) with the spin infection method. As control to Foxp3-transduced T cells (Foxp3-T cells), virus containing pMX-IRES-EGFP empty vector was also infected to mouse CD25−CD4+ T cells to generate empty vector-transduced T cells (Mock-T cells). EGFP-positive Foxp3-T or Mock-T cells were purified by BD FACSAria cells sorting system (BD Biosciences, CA, USA) at over 99% purity and total RNAs were isolated from both of the cells by using TRIZOL reagent (Invitrogen Life Technologies, CA, USA).

Normalized Representational Differential Analysis (N-RDA)

RNA samples of Foxp3-T or Mock-T cells were applied to N-RDA using the methods described in WO 02/103007 A1, the entire contents of which are incorporated herein by reference.

Gene Chip Analysis

Non-stimulated or antibody-stimulated Foxp3-T cells or Mock-T cells were prepared for gene chip analysis. In the 1^{st} round of gene chip analysis, the following 4 populations were prepared, M−: non-stimulated Mock-T cells, M+: stimulated Mock-T cells, F−: non-stimulated Foxp3-T cells, F+: stimulated Foxp3k-T cells. In the 2^{nd} round, antibody-stimulated Foxp3-T or Mock-T cells were prepared at different time points (6 hr-stimulation and 24h-stimulation) along with non-stimulated cells.

Total RNAs were extracted from the cells using Trizol (Invitrogen Life Technologies, CA, USA) and further purified with RNeasy columns (QIAGEN, Valencia, Calif., USA). To obtain an adequate amount of cRNA, a two-cycle amplification method to make biotinylated complementary RNA (cRNA) was carried out for the GeneChip probe. First, Double-stranded cDNA was prepared from 100 ng of total RNA using Super-Script Choice System (Life Technologies, Inc.) with T7-Oligo(dT) promoter primer. After purification of cDNA by ethanol precipitation, in vitro transcription (IVT) was carried out using MEGA-script T7 Kit (Ambion), and then cRNA was cleaned up with RNeasy columns (Qiagen) (First Cycle of Amplification). As a second cycle of amplification of cRNA, a second cycle 1^{st} strand cDNA was synthesized from 400 ng of cRNA using Super-Script II (Life Technologies, Inc.) with random primers, and a 2^{nd} strand cDNA was synthesized with T7-Oligo(dT) promoter primer. After purification by ethanol precipitation, the second cycle IVT was carried out with an RNA Transcript Labeling kit containing biotinylated UTP and CTP (Enzo Diagnostics), and then labeled cRNA was purified with RNeasy columns. This labeled cRNA was fragmented and then hybridized to Affymetrix GeneChip MOE430A arrays. According to the EukGE-WS2 protocol, the probe arrays were washed and stained with streptavidin-phycoerythrin and biotinylated goat anti-streptavidin on an Affymetrix fluidics station. Fluorescence intensities were captured with a Hewlett Packard confocal laser scanner. All quantitative data were processed using the Affymetrix GeneChip software, MAS5.0.

Results

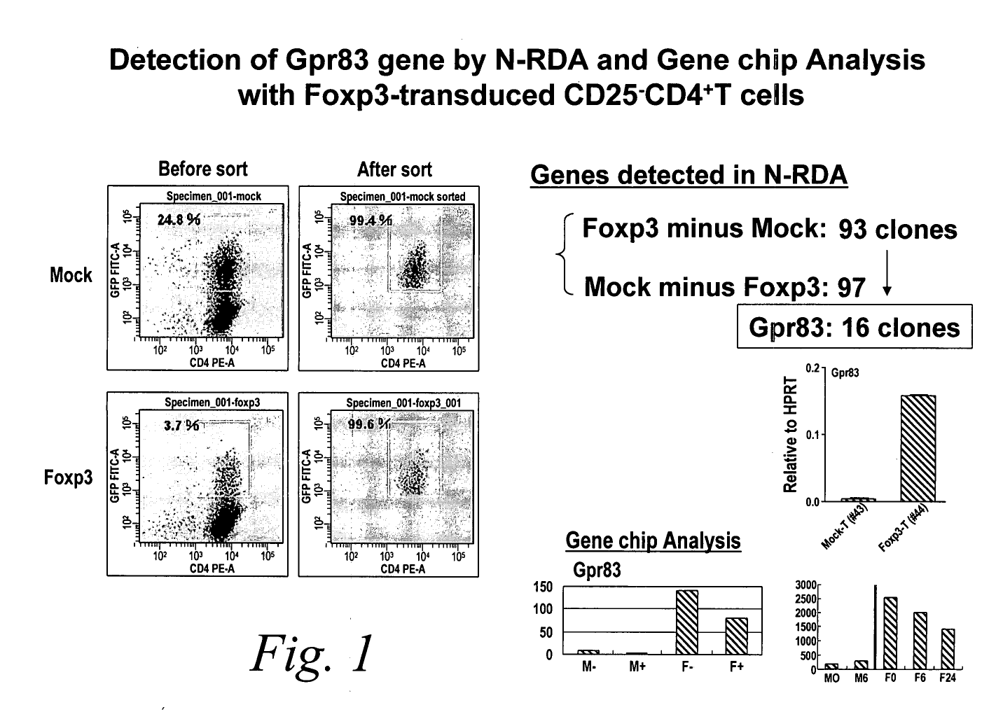

After the 4^{th} round of subtraction of the complementary DNA (cDNA) of N-RDA, 93 clones were identified as molecules exclusively expressed in the Foxp3-T cells, and among those, mGPR83 (GenBank Accession No.: gi:6753987 (NM_{—}010287)) was the most frequently detected (16 clones in 93). This high frequency meant not only that mGPR83 was exclusive expressed in Foxp3-T cells but also the amount of expression was relatively high. A quantitative real time polymerase chain reaction (PCR) by using the same RNA samples as used in the N-RDA confirmed the excusive expression of mGPR83 in Foxp3-T cells because mGPR83 was consistently detected at high level of expression only in Foxp3-T cells. Consistent with the results of N-RDA, GeneChip analysis also revealed that mGPR83 is a specific molecule to Foxp3-T cells both in the 1^{st} analysis and the 2^{nd} one. The expression levels tended to be down-regulated after the T cell receptor (TC R)-mediated stimulation of Foxp3-T cells but the mGPR83 was not detected in control Mock-T cells even though the cells were stimulated. Collectively, the foregoing data (depicted in FIG. 1) indicates that mGPR83 was specifically induced at a substantial level under the existence of Foxp3 transcriptional factor and never induced in non-Treg cell populations.

### Example 2

### mGPR83 Expression is Exclusive to CD25+CD4+ Treg Cells as Confirmed by Quantitative Real Time PCR

Aim of Experiment

Transcriptome analysis by N-RDA and Genechip repeatedly detected mGPR83 as a CD25+CD4+ Treg specific gene. In order to validate and confirm whether or not mGPR83 is a molecular target for Treg cells, quantitative real time PCR experiments were performed using a mouse lymphocyte panel and freshly isolated or gene-transduced T cells.

Materials and Methods

Cells

An RNA panel of mouse lymphocyte populations such as B220+ B cells, CD11b+Ly6G-macrophages, bone marrow derived dendritic cells (BM-DC), CD4+ T cells, CD8+ T cells, CD4+ helper T cell type I (Th1) and CD4+ helper T cell type II (Th2), were prepared or generated from mice (Japan SLC. Inc., Shizuoka, Japan). CD25−CD4+ T and CD25+CD4+ T cells were magnetically prepared from BALB/c mice (Japan SLC. Inc., Shizuoka, Japan) according to the manufacturer's protocol (CD4+ T cell isolation kit and CD4+CD25+ Regulatory T Cell Isolation Kit, Miltenyi Biotech GmbH, Bergisch Gladbach, Germany). Mock-T or Foxp3-T cells were prepared by the same method described in Example 1. In some analyses, the cells were further stimulated with various combinations of antibodies such as anti-CD3 (BD Biosciences, San Jose, Calif., USA), anti-CD28 (BD Biosciences, San Jose, Calif., USA), anti-CTLA-4, and recombinant murine interleukin-2 (R&D systems Inc., Minneapolis, Minn., USA). For the real time PCR, the cells were collected and homogenized in TRIZOL reagent (Invitrogen Life Technologies, CA, USA), thereafter RNAs were isolated according to the manufacturer's protocol.

Quantitative Real Time PCR

The expression levels of mGPR83 in the cells were evaluated by quantitative real time PCR in which the amount of mGPR83 was internally compared with the level of a house keeping gene, hypoxanthine guanine phosphoribosyl transferase (HPRT). First, RNA samples were converted into complementary DNA (cDNA) with an RNA PCR kit (TAKARA Bio Inc. Otsu, Shiga, Japan) according to manufacture's protocol. The following primers were used in the PCR to cDNA samples: For HPRT, 5′-CAGGCCAGACTTTGTTGGAT-3′ and 5′-TTGCGCTCATCTTAGGCTTT-3′ were used as forward and reverse primer, respectively. For mGPR83, 5′-CATCTGGGTCATGGCTACCT-3′ and 5′- GCCAGGTCCAGATACTTCCA-3′ were used as forward and reverse primer, respectively. All primers were designed using the WWW-based software, Primer 3 (Whitehead Institute for Biomedical Research, Cambridge, Mass., USA) to avoid the non-specific amplification of RNA.

Reaction mixtures composed of cDNA template, primers, uracil DNA glycosylase (Invitrogen, CA, USA), QuantiTect SYBR Green PCR Master Mix (QIAGEN, Valencia, Calif., USA) and appropriate amount of distilled water were applied to the ABI PRISM 7700 Sequence Detection System (Applied Biosystems, Foster, Calif., USA) in which the ABI PRISM 7700 Sequence Detector Software automatically quantified the expression levels.

Results

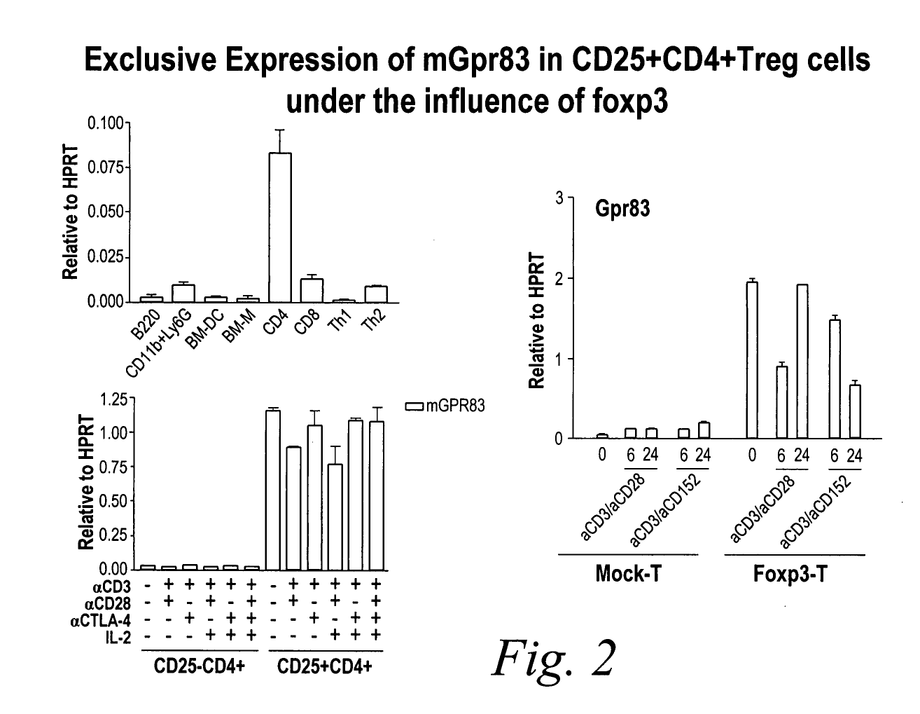

As indicated in FIG. 2, among the lymphocyte panel, only CD4+ T cells, which include CD25+CD4+ Treg cells, gave high expression of mGPR83. CD4+ T cells are classified into two populations according to expression of CD25 and mGPR83 was detected only in CD25+CD4+ T cells that are equal to CD25+CD4+ Treg cells. The level of expression did not fluctuate when several combinations of antibody stimulation were used. Importantly, mGPR83 was never induced in CD25−CD4+ T cells. Because mGPR83 showed a tendency of down-regulation after antibody-mediated stimulation of Foxp3-T cells, mGPR83 was quantified on the Foxp3-T cells stimulated with anti-CD3 and anti-CD28 or anti-CTLA-4 (CD152). Stimulation with anti-CD28 transiently down-modulated the mGPR83 expression. On the other hand, stimulation with anti-CTLA-4 seems to down regulate mGPR83 gradually. However, it was evident from all of the experiments that mGPR83 was actually expressed on freshly isolated CD25+CD4+ Treg cells and Foxp3-transduced T cells with dramatic specificity (see FIG. 2).

### Example 3

### mGPR83 Quantification to Determine the Lymphoid System Specificity of mGPR83

Aim of Experiment

To select a potentially druggable molecular target, the tissue distribution of the molecule is quite important from the specificity point of view. In this experiment, the tissue distribution of mGPR83 was evaluated using freshly isolated mice tissues.

Materials and Methods

Various tissues were excised out from 7wk old BALB/c female mice and quickly frozen with dry ice to avoid RNA degradation. Total RNAs were extracted from the tissues using Trizol (Invitrogen Life Technologies, CA, USA) after homogenization of the tissue. The cDNAs for real time PCR template were generated by the same protocol used in the Example 2. The PCR reactions were performed also using the methods described in Example 2. HPRT was used as an internal control to quantify the mGPR83. As a reference for mGPR83, Foxp3 was also evaluated. The primer sets for HPRT and mGPR83 were the same as those used in Example 2. For Foxp3, 5′-GGAGCTGGAAAAGGAGAAGC-3′ and 5′-GCTACGATGCAGCAAGAGC-3′ were used as forward and reverse primers, respectively.

Results

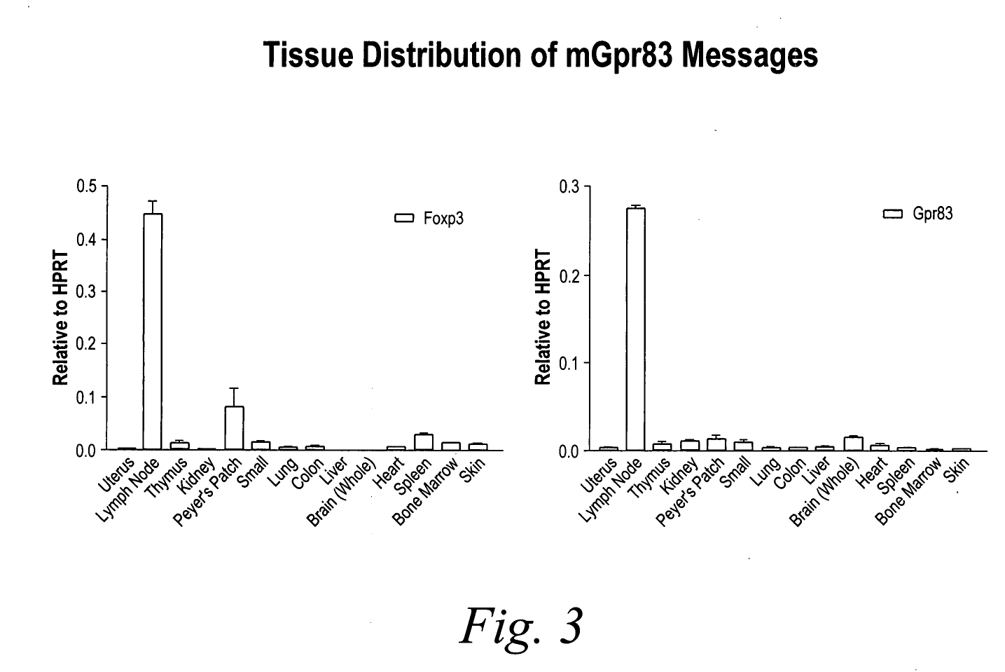

As indicated in FIG. 3, lymph node that is abundant of Foxp3 message was detected as a major site of expression of mGPR83. Although brain gave expression of mGPR83 at a certain level, a broad distribution was not seen.

### Example 4

### Human GPR83 is Also Predominantly Expressed in CD4+CD25+ Human Treg Cells

Aim of Experiment

Through the experiments described above with the mouse system, it was evident that mGPR83 was mainly expressed in the lymphoid system in the body, and specifically that CD25+CD4+ Treg cells were unique cells to bear mGPR83. To confirm the pattern of expression of human GPR83 the following analysis was performed.

Materials and Methods

Preparation of human CD4+CD25⁻ and CD4+CD25+ T cells Peripheral blood mononuclear cells (PBMC) were isolated from 150 ml heparinized venous blood from a healthy volunteer with Ficoll-Paque (Amersham Pharmacia Biotech) centrifugation. CD4⁺CD25⁻ and CD4⁺CD25⁺ T cell fractions were collected from PBMC with MACS separation columns using a human CD4+CD25+ regulatory T cell isolation kit (Myltenyi Biotec) according to the manufacturer's instructions. Both isolated cell fractions were applied to FACSAria (BD Biosciences), further purified, and 99 % pure CD4+CD25− and 91 % pure CD4+CD25+ T cell fractions were obtained.

Preparation of cDNA

The isolated cell fractions were solubilized in Isogen solution (Nippon Gene), and total RNA was purified. The RNA samples were mixed with oligo(dT) 12-18 primer (Invitrogen) and converted to the first strand cDNA using the superscript II reverse transcriptase (Invitrogen).

Quantitative Real Time PCR

For quantitative confirmation of gene expression, real time PCR was performed using an ABI PRISM 7900 Sequence Detection System (Applied Biosystems). The cDNA samples were amplified by introducing TaqMan PCR Master Mix, Assay-on-Demand primers and probes designed by Applied Biosystems (Foxp3:Hs00203958_ml, GPR83/GPR72:00173906_ml). Each expression level was standardized by the level of human house keeping gene, beta-actin, quantified using the standard primers and the TaqMan probe (Applied Biosystms). Both gene expression levels in CD4+CD25+ T cells were shown as fold changes compared with the levels in CD4+CD25− T cells.

Results

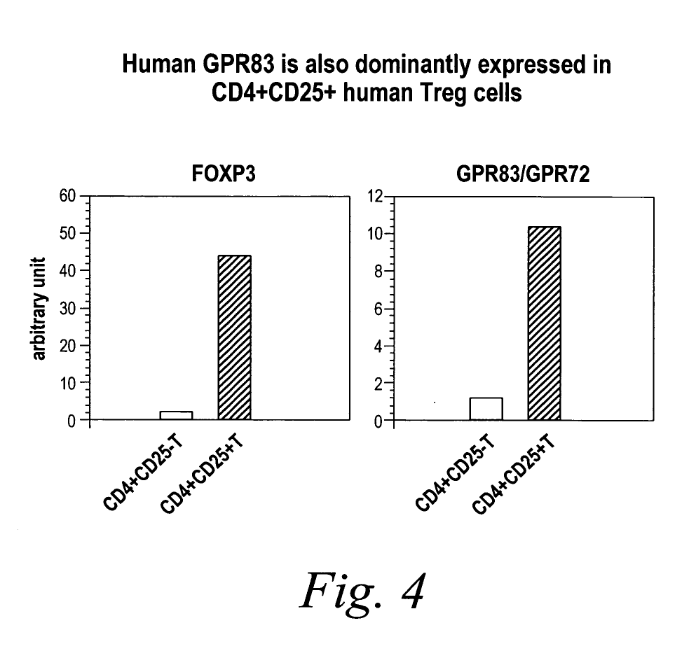

As indicated in FIG. 4, the expression of both human Foxp3 and human GPR83/GPR72 genes measured by real time PCR was significantly higher in the CD4⁺CD25⁺ than in the CD4+CD25− T cell fraction. GPR83 was specifically expressed in human CD25⁺CD4⁺ T cells (as was the case for mouse Treg cells).

### Example 5

### Human GPR83 is Also Predominantly Expressed in Human Treg Cells

Aim of Experiment

Through the experiments described above with the mouse system, it was evident that mGPR83 was mainly expressed in the lymphoid system in the body, and specifically that CD25⁺CD4⁺ Treg cells were unique cells to bear mGPR83. To confirm the pattern of expression of human GPR83 the following analysis was performed.

Materials and Methods

Cells

Human peripheral blood was drawn from healthy volunteers, and the lymphocytes were colleted by gradient centrifugation with Ficoll-Paque Plus reagent (Amersham Biosciences, Piscataway, N.J., USA). After enrichment of CD25⁺CD4⁺ T cells and CD25⁻CD4⁺ T cells by magnetic sorting system (CD4⁺CD25⁺ Regulatory T Cell Isolation Kit Human, Miltenyi Biotech GmbH, Bergisch Gladbach, Germany), cell populations were further sorted into CD25^{high}CD4+ T cells, CD25^{low}CD4+ T cells, and CD25⁻CD4⁺ T cells at high purity.

Quantitative Real Time PCR

The expression levels of GPR83 in the cells were evaluated by quantitative real time PCR in which the amount of genes of interest was compared with a control gene, beta-actin.

First, RNA samples were converted into complementary DNA (cDNA) using an RNA PCR kit (TAKARA Bio Inc. Otsu, Shiga, Japan) according to the manufacture's protocol. Specific primer sets to measure the levels of FOXP3, GPR83 and beta-actin were obtained from Applied Biosystems (Foster, Calif., USA ) and were used to run quantitative Taqman PCR according to the manufacturer's protocol.

Results

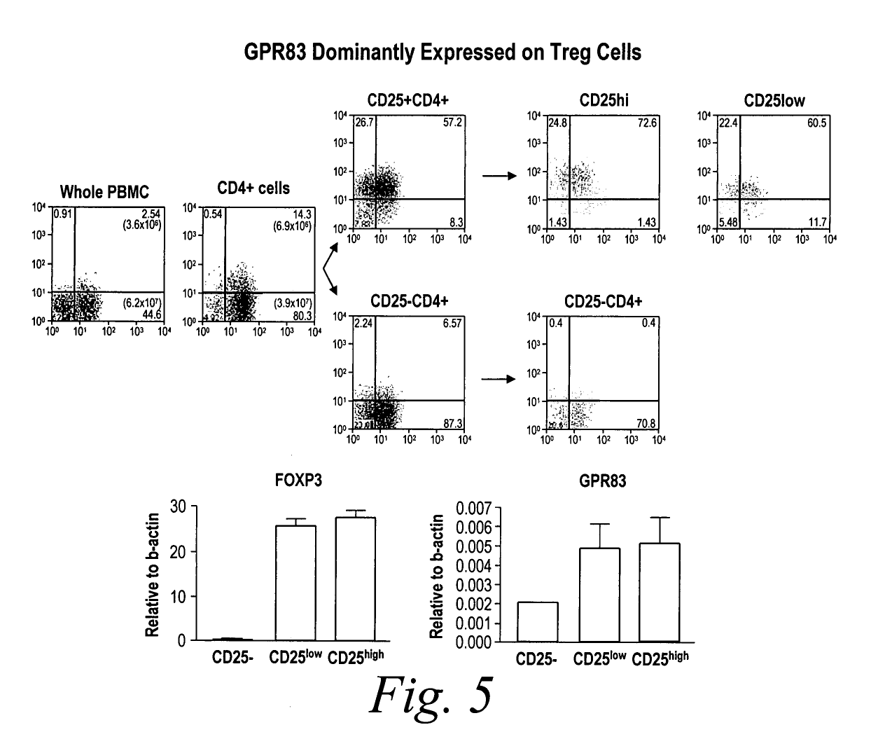

As indicated in FIG. 5, FOXP3 was detected both in CD25^{high} and CD25^{low} populations but not in the CD25⁻CD4⁺ T cells. Human GPR83 showed the same pattern of expression the one for FOXP3.

### Example 6

### Tissue Distribution of hFOXP3 and hGPR83

Aim of Experiment

The purpose of the experiment described below was to evaluate the distribution of human GPR83 in vatiouse human tissue samples.

Materials and Methods

Samples

RNA samples from various human tissues were purchased from BD Biosciences (San Jose, Calif., USA).

Quantitative Real Time PCR

The same methods described in Example 5 were used.

Results

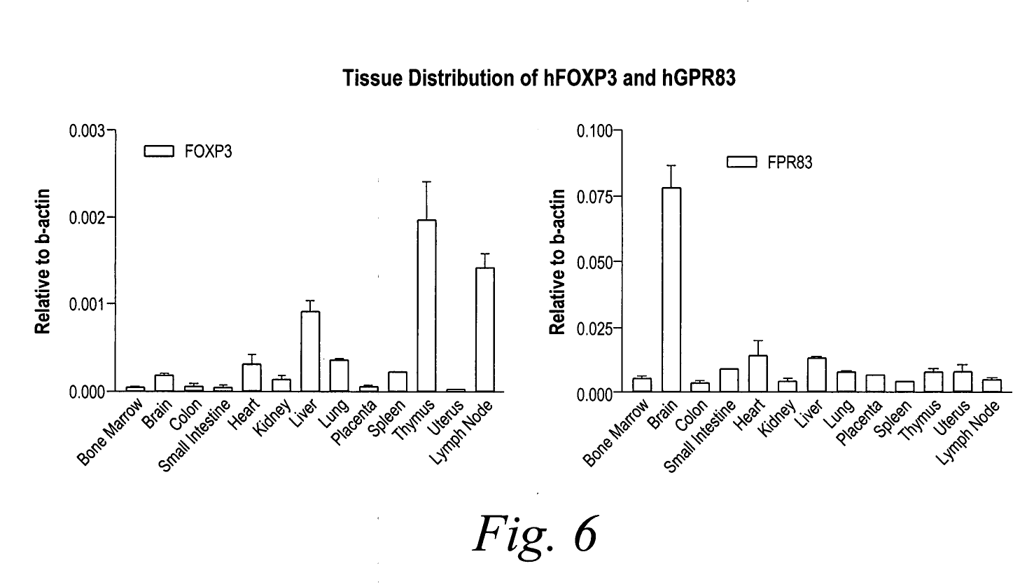

As indicated in FIG. 6, contrary to the site of FOXP3 expression (lymphoid tissues), the major site of human GPR83 expression was the brain. Lymphoid tissues did not give high expression of GPR83. Because the GPR83 level of expression directly depends on the frequency of GPR83-expressing cells in the tissues, low expression of GPR83 in lymphoid tissues was not surprising.

### Example 7

### Generation of Antibodies Against Murine GPR83 and Confirmation of The Specificity of Murine GPR83 Expression on Treg Cells at the Protein Level

Aim of Experiment

The purpose of this experiment was to confirm the expression of mGPR83 at the protein level. For this purpose, monoclonal antibodies against mGPR83 were generated.

Materials and Methods

Antigen Preparation

In order to generate monoclonal antibodies against mGPR83, a mGPR83 protein N-terminally fused with glutathione S transferase (GST) (GST-1 exmGPR83) was first generated. The 1^{st} extracellular domain of mGPR83 was subcloned into pGEX4T3 vector (Amersham Pharmacia Biotech, Piscataway, N.J., USA) using primers, 5′- CGCGTCGACGCCACCatgaaggttcctcctgtcct-3′ (forward) and 5′-GCGGGCGGCCGCtttcaccgtggggttctggg-3′ (reverse) (pGEX4T3-1 exmGPR83). E.coli, JM109 strain (TAKARA Bio Inc. Otsu, Shiga, Japan) was transformed with pGEX4T3-1 exmGPR83. After protein induction by Isopropyl-β-D(−)-thiogalactopyranoside (IPTG, Wako Pure Chemical Industries Ltd., Osaka, Japan), the bacteria was solubilized with salkosyl (Sigma-Aldrich, St. Louis, Mo., USA) and thereafter the GST-1 exmGPR83 fusion proteins were purified by using glutathione sepharose 4B column (Amersham Pharmacia Biotech, Piscataway, NJ, USA).

Immunization of Animal

WKY rats (Charles River Japan, Yokohama, Kanagawa, Japan) were immunized with 2 μg of purified GST-1 exmGPR83 fusion proteins with TiterMax Gold (CytRx Corporation, Norcross, Ga) as adjuvant. After the sequential immunizations, lymph node cells of rats were fused with the P3x64Ag8.653 myeloma cell line by polyethrene glycol (PEG, Behringer-Ingerheim, Germany) to generate hybridoma cells. After HAT selection (Invitrogen Life Technologies, CA, USA), hybridomas were cloned by limiting dilution after evaluation of the reactivity to mGPR83-transduced B300 cells. Clone 27.31 (rat IgG2b) was one of the selected clones and was used for further experiments. For FACS analysis, 27.31 was labeled with a fluorochrome, Alexa Fluor 647 (Alexa Fluor 647 Monoclonal Antibody Labeling Kit, Invitrogen, CA, USA), according to the manufacturer's protocol.

FACS Analysis of CD25⁺CD4⁺ Regulatory T Cells

Freshly isolated CD25⁺CD4⁺ T cells and CD25⁻CD4⁺ T cells were separately cultured in the presence of IL-2 (50 U/ml) and dexamethasone (10 nM, Sigma-Aldrich, St. Louis, Mo., USA) for 18 hours. During the last 1-hour, the cells were incubated with FITC-conjugated anti-mCD4 (RM4-5, 5 μg/ml, BD Biosciences, San Jose, Calif., USA), PE-conjugated rat anti-mCD25 (PC61.5, 4 μg/ml, eBIOscience, San Diego, Calif., USA), and Alexa Fluor 647-conjugated 27.31 (10 μg/ml). After washing the cells, the cells were analyzed using the BD FACSCalibur flow cytometer (BD Biosciences, San Jose, Calif., USA)

Results

Even though the 27.31 mAb specifically detected mGPR83 on the B300 cells transduced with mGPR83, little positive staining of mGPR83 was observed on the freshly isolated CD25⁺CD4⁺ Treg cells using this monoclonal antibody. Therefore, an experimental technique was designed (based on the techniques described in Harrigan M et al. Mol Cell Biol., 9: 3438-3446, 1989 and Chen X et al. E J Immunol 34: 859-869, 2004) to induce mGPR83. The technique employs the incubation of CD25⁺CD4⁺ T cells with IL-2 and glucocorticoids.

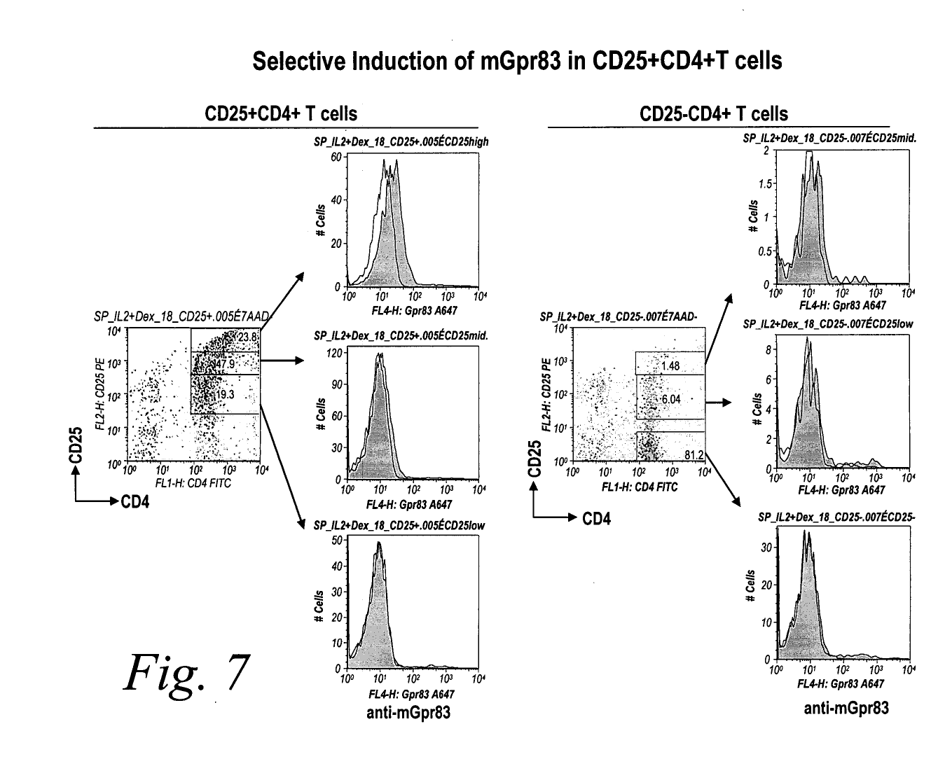

Only Treg cells responded to IL-2 and a glucocorticoid, dexamethasone, with an increase in the size of the cells (blastic change) and a further induction of CD25 and CD4 (CD25^{high}CD4^{high} population in the left chart), while no phenotypic change was observed in CD25⁻CD4⁺ T cells. Interestingly only blastic CD25⁺CD4⁺ Treg cells gave positive staining with 27.31, while no 27.31-positive staining was seen in the CD25⁻CD4⁺ T cells. Thus, the foregoing experiments (graphically depicted in FIG. 7) demonstrate that mGPR83 is expressed exclusively on CD25⁺CD4⁺ Treg cells not only at the mRNA level but also at the protein level.

### Example 8

### GPR83 Ligand Activity Was Detected In Peptidic Fraction From Mouse And Porcine Brain

CRE-PLAP Reporter Cells Engineered with the GPR83 Gene

The CRE-PLAP reporter gene, which contains a tandem tetramer of the cyclic AMP response element (CRE) cloned upstream of a fragment of human vasoactive intestinal peptide gene promoter functionally linked to a human secreted-type placental alkaline phosphatase (PLAP) gene, was constructed in a retrovirus vector as described in Chen, W. et al. (1995) Anal. Biochem. 226, 349-354, Durocher, Y. et al. (2000) Anal. Biochem. 284, 316-326., and Goto, M. et al. (1996) 49(5), 860-873. To obtain stable CRE-PLAP expressing HEK293 cell lines, cells were transduced with a retrovirus vector containing the CRE-PLAP expression unit. The transduced cell lines were analyzed using a PLAP assay and the best clone was used as a host cell line for the transfection with the GPR83 gene. The GPR83 gene, which was obtained by PCR using human brain cDNA as a template, was also introduced by the same procedure.

Ligand Screening Assay with CRE-PLAP Reporter Cells

HEK293/CRE-PLAP/GPR83 cells were seeded with 100 μl of Dulbecco's Modified Eagle's Medium/Ham's Nutrient Mixture F12 (DMEM/F12) medium supplemented with 10% (v/v) fetal bovine serum at 1×10⁴ cells per well in 96-well plates and incubated for 24 h at 37° C. in a CO₂ incubator (5% CO₂). The cells were then stimulated by addition of 10 μl reconstituted sample and 10 μl of 10 μM Forskolin/DMEM per well. Following incubation at 37° C./5% CO₂ for 24 h, 5 μl of the culture media per well was transferred to white 384 Well Plate (Nalge Nunc International) with 20 μl of assay buffer (280 mM Na₂CO₃-NaHCO₃, 8 mM MgSO₄, pH 10) and 25 μl of Lumiphos 530 (Lumigen) and incubated at Room Temperature for 2 h. The level of expressed PLAP was quantified on a Fusion plate reader (Perkin Elmer).

Partial Purification of the Crude Natural Ligand of GPR83

For the mouse brain-derived fractions, approximately 60 g of brains without cerebellum were homogenized by a blender in 10× volume of 70% (v/v) acetone, 1M acetic acid and 20 mM HCl and then centrifuged at 15000× g for 30 min at 4° C. The resultant supernatant was collected and extracted twice with diethyl ether. The aqueous phase was centrifuged again and the supernatant was loaded onto two 10 g cartridge C18 column, HF MEGA BE-C 18 (VARIAN), pre-equilibrated with 0.1% TFA. Cartridges were washed with 40 ml of 0.1% TFA, and then eluted with 50% CH₃CN/0.1% TFA. The eluate was lyophilized, re-dissolved in 1M acetic acid and applied to HPLC. Step 1:¼ of the extract was loaded onto a C18 reversed-phase HPLC column, YMC ProC18 (4.6 mm×250 mm), pre-equilibrated with 0.1% TFA. The loaded sample was eluted with a 50-min linear gradient of 24-48% CH₃CN in 0.1% TFA at a flow rate of 1 ml/min. Fractions were collected at 1-min intervals. ≠1% of each fraction was subjected to the CRE-PLAP assay in order to determine whether or not the fraction had an effect on the CRE-PLAP reporter cells transduced with GPR83. Step 2: The active fractions were pooled, diluted 4-fold with 0.1% TFA, and loaded onto a diphenyl reversed-phase column, Vydac 219TP54 (4.6 mm×250 mm), preequilibrated with 0.1% TFA. A 29.4-51% gradient of CH₃CN in 0.1% TFA was applied over 50 min at a flow rate of 1 ml/min. Fractions were collected at 1-minute intervals and 5% of each fraction were assayed. The procedure of Homogenization and extraction (Step1 and Step2) was repeated six times, so a total of 360 g of mouse brain was processed. Step 3: All of the active fractions were pooled, diluted 4-fold with 0.1% TFA, and loaded onto a C18 reversed-phase column, Vydac 218TP54 (4.6 mm×250 mm), pre-equilibrated with 0.1% TFA. A 30-54% gradient of CH₃CN in 0.1% TFA was applied over 50 minutes at a flow rate of 1 ml/min. Fractions were collected at 1-minute intervals and 10% of each fraction were assayed. The active fraction was used as a crude ligand of GPR83.

For the porcine-derived fractions, almost the same procedure was performed, except the initial brain volume was 90 g and ⅙ of the sample was subjected to HPLC and then approximately 3% of each fraction was subjected to the CRE-PLAP assay.

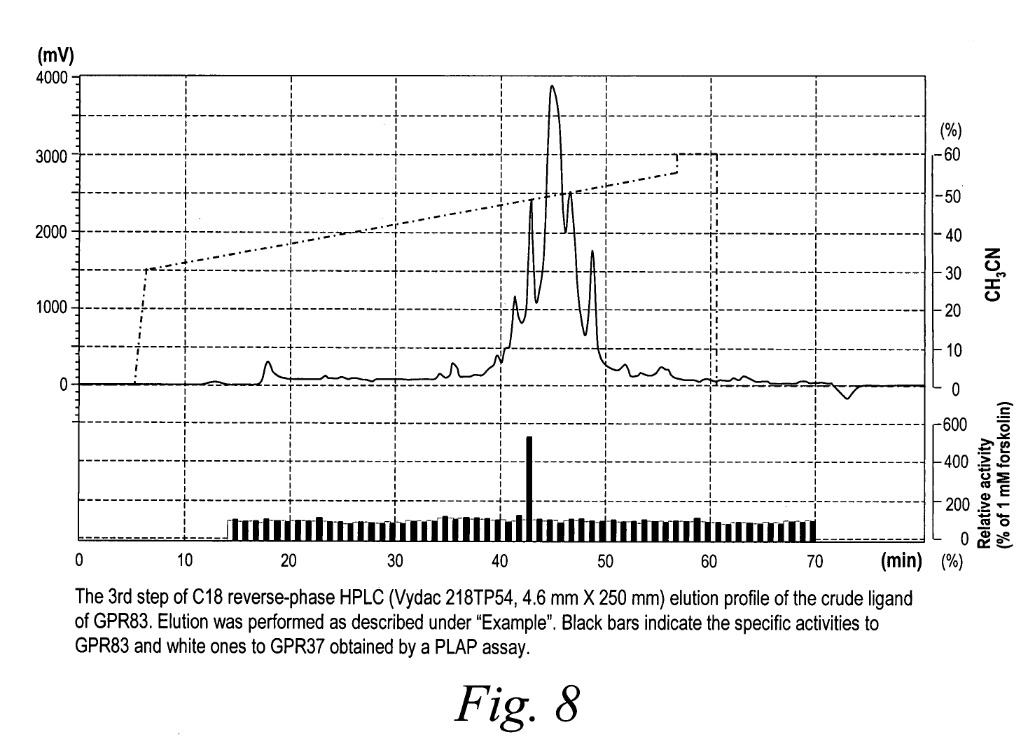

The results of the analysis are shown in FIG. 8. As demonstrated in FIG. 8, substantial ligand activity is detected in the mouse brain derived active fraction.

Characterization of the Crude GPR83 Ligand of GPR83

The sample was prepared as described above.

To compare with a peptide ligand, the galanin receptor 2(GAL2R) was used as a control (because brain extracts contain a lot of galanin). Each fraction was treated as follows.
1) Control. Reconstituted by 50 μl of 0.1% TFA and dried and dissolved with 24 μl of 0.1% TFA. Then 10 μl/ea (to GPR83 expressing cells and Galanin receptor 2 (Gal2R) expressing cells) was added in the PLAP assay. The specific activities of Gal2R and GPR83 were then determined.
2) Acid treatment. Reconstituted by 50 μl 5M HCl and incubated at 55° C. for 12 h. Then dried and dissolved with 24 μl of 0.1% TFA again. Then 10 μl/ea was added in the PLAP assay. Acid treatment extinguished both galanin activity and GPR83 specific activity.
3) Proteinase K (ProK) treatment. Reconstituted by 50 μl of ProK sol.(100 μg/ml ProK/PBS) and incubated at 55° C. for 1 h. To inactivate ProK (because ProK inhibits the PLAP assay) the sample was incubated at 90° C. for 15 min. Then dried and dissolved with 24 μl of 0.1% TFA. Then 10 μl/ea was added in the PLAP assay. The ProK treatment extinguished both galanin activity and GPR83 specific activity.
4) Heat treatment. Reconstituted by 50 μl H2O and to standardize with the above experiment, incubated at 55° C. for 1 h. The sample was then incubated at 90° C. for 15 minutes and then dried and dissolved with 24 μl of 0.1 % TFA. Then, 10 μl/ea was added in the PLAP assay. The heat treatment attenuated GPR83 specific activity by half but did not change the galanin activity.

### Example 9

### In Vitro Analysis of Treg Cell Function

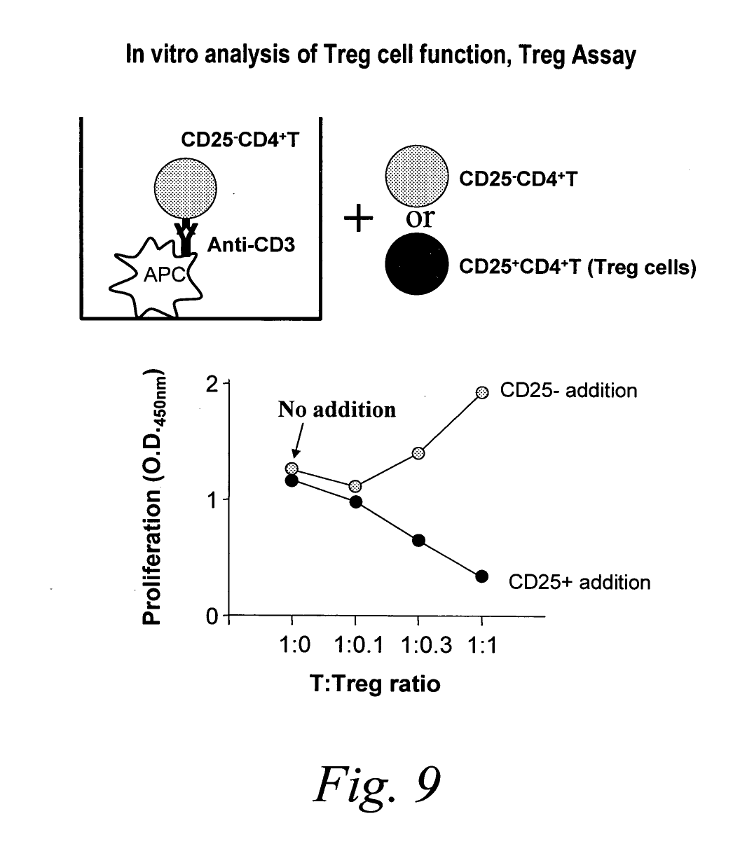

This assay may be performed as described in (Itoh M. et al. (1999) J. Immunol 162: 5317-5326, the contents of which are hereby incorporated herein by reference. Briefly, mouse CD25⁻CD4⁺ T or CD25⁺CD4⁺ T cells were magnetically prepared from 7 wk old BALB/c female mice (Japan SLC. Inc., Shizuoka, Japan) according to the manufacturer's protocol (CD4+ T cell isolation kit and CD4+CD25+ Regulatory T Cell Isolation Kit, Miltenyi Biotech GmbH, Bergisch Gladbach, Germany). Obtained CD25⁻CD4⁺ T cells (Responder, 1×10⁵ cells) were co-cultured with mitomycin C (Sigma-Aldrich, St. Louis, Mo., USA)-treated non-CD4 splenocytes (1×10⁵ cells) in the presence of soluble anti-CD3 mAb (145-2C11, 10 μg/ml , BD Biosciences, San Jose, Calif., USA). CD25⁺CD4⁺ Treg cells (gray) or CD25⁻CD4⁺ T cells (blck) were added to the culture as regulator cells or control of regulators, respectively, in a different Responder/Regulator ratio (from 1:0 to 1:1). Because CD25⁺CD4⁺ Treg cells are anergic to any TCR-mediated stimulation (i.e., they are non-proliferative to TCR-mediated stimulation), the proliferation value of the culture is derived only from CD25⁻CD4⁺ responder T cells. In this situation, if Treg cells are added to the culture, Treg number-dependent inhibition of proliferation will be observed, while no inhibition but increased proliferation may be seen in the CD25⁻CD4⁺ T cell addition (see FIG. 9).

### Example 10

### Mouse Brain Derived Ligand for GPR83 Enhances the Activity of CD25⁺CD4⁺ Treg Cells

Aim of Experiment

In order to determine the biological effect of the ligand of GPR83 on CD25⁺CD4⁺ Treg cells, in vitro a Treg assay was performed in the presence of a mouse brain-derived active fraction (generated as described in Example 8).

Materials and Methods

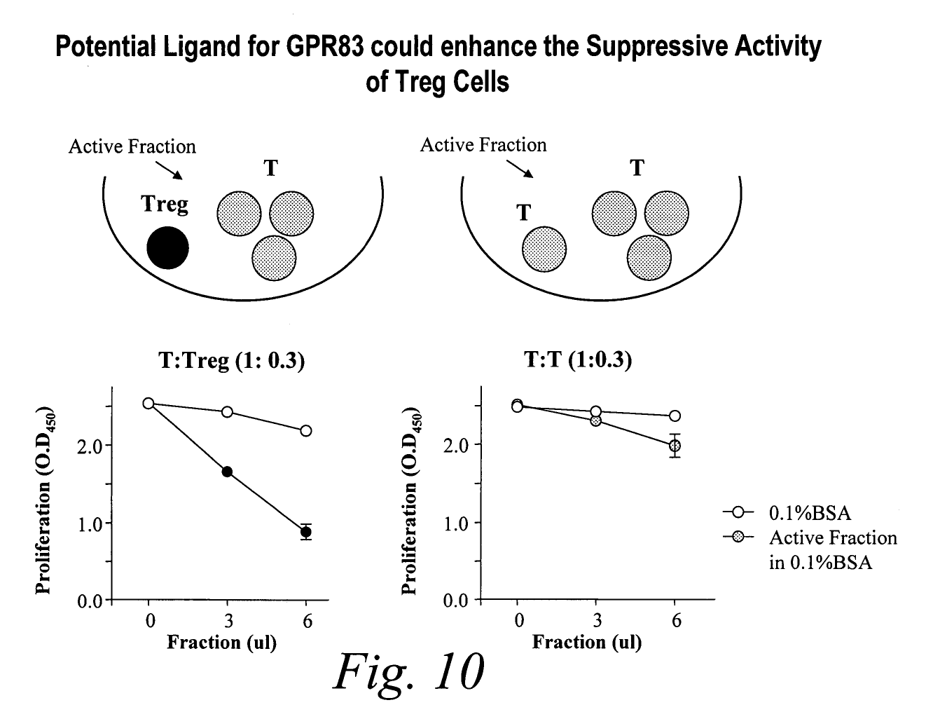

Mouse CD25⁻CD4⁺ T or CD25⁺CD4⁺ T cells were magnetically prepared from 7 wk old BALB/c female mice (Japan SLC. Inc., Shizuoka, Japan) according to the manufacturer's protocol (CD4+ T cell isolation kit and CD4+CD25+ Regulatory T Cell Isolation Kit, Miltenyi Biotech GmbH, Bergisch Gladbach, Germany). The resulting CD25⁻CD4⁺ T cells (Responder, 1×10⁵ cells) were co-cultured with mitomycin C (Sigma-Aldrich Corporation, St. Louis, Mo., USA) -treated non-CD4 splenocytes (1×10⁵ cells) in the presence of soluble anti-CD3 mAb (145-2C11, 10 μg/ml, BD Biosciences, San Jose, Calif., USA). CD25⁺CD4⁺ Treg cells (FIG. 10, left panel) or CD25⁻CD4⁺ T cells (FIG. 10, right panel) were added to the culture as regulator cells or control of regulators, respectively, at a ratio of 1:0.3 of Responder/Regulator ratio. In order to evaluate the biological effect of the ligand for GPR83, mouse brain derived fraction was added to the cultures (3 μl or 6 μl, FIG. 10, closed circles). As a control for ligand fraction, 0.1 % bovine serum albumin (BSA) water that is a solvent of the ligand fraction was added to the cultures (3 μl or 6 μl, FIG. 10, open circles). During the last 4 hours of the 96-hr culture, WST-8 (Cell Count Reagent SF, Nacalai Tesque, Inc., Kyoto, Japan) reagent was added to measure the proliferative extent of T cells according to the manufacturer's protocol. All cultures were performed in the presence of S-clone SF-O3 complete serum free media (Sanko Junyaku Co., Ltd., Tokyo, Japan) supplemented with HEPES (20 mM, Dojinbo Laboratories, Kumamoto, Japan), kanamycin sulfate (100 μg/ml, Invitrogen, CA, USA), 2-mercaptethanol (55 μM, Invitrogen, CA, USA) and 10% fetal-calf serum (HyClone, Utha, USA).

Results

As indicated in FIG. 10, proliferation of responder CD25⁻CD4⁺ T cells was significantly inhibited by the addition of the mouse brain derived active fraction in a dose dependent manner while no inhibition was observed in the control. In addition, the mouse brain derived active fraction had no effect on the CD25⁻CD4⁺ T cell culture. The foregoing data (graphically depicted in FIG. 10) demonstrate that the mouse brain derived active fraction (containing a possible ligand for GPR83) specifically stimulates CD25⁺CD4⁺ T cells and augments their immunoregulatory activity.

### Example 11

### Mouse Brain Derived Ligand for GPR83 Activates CD25⁺CD4⁺ Treg Cells to Produce Cytokines

Aim of the Experiment

In addition to the in vitro Treg assay (T cell proliferation assay) described above, the effect of the mouse brain derived GPR83 ligand was evaluated by measuring the cytokine production from Treg cells. Because IL-10 (Hara M et al. J Immunol. 166: 3789-3796, 2001, Kingsley C I et al. J Immunol. 168: 1080-1086, 2002) and IFN-gamma (Fallarino F et al. Nat Immunol. 4: 1206-1212, 2003) are known to be critical cytokines for the Treg cell immunoregulatory activity, these cytokines were measured in the presence or absence of the mouse brain derived active fraction (containing a GPR83 ligand).

Materials and Methods

Magnetically purified CD25⁺CD4⁺ Treg cells were stimulated with plate-bound anti-CD3 (145-2C11, 10 μg/ml, BD Biosciences, San Jose, Calif., USA), soluble anti-CD28 (37.51, 2 μg/ml, BD Biosciences, San Jose, Calif., USA) and recombinant murine IL-2 (200 U/ml) with or without the active fraction or control 0.1% BSA. Twenty-four hours or 48 hours later, the culture supernatants were collected to measure the amount of IL-10 (FIG. 10, top part) and IFN-gamma (FIG. 10, bottom part) using an ELISA kit (DuoSet ELISA Development kit, R&D Systems, Inc. Minneapolis, Minn., USA) according to the manufacturer's protocol.

Results

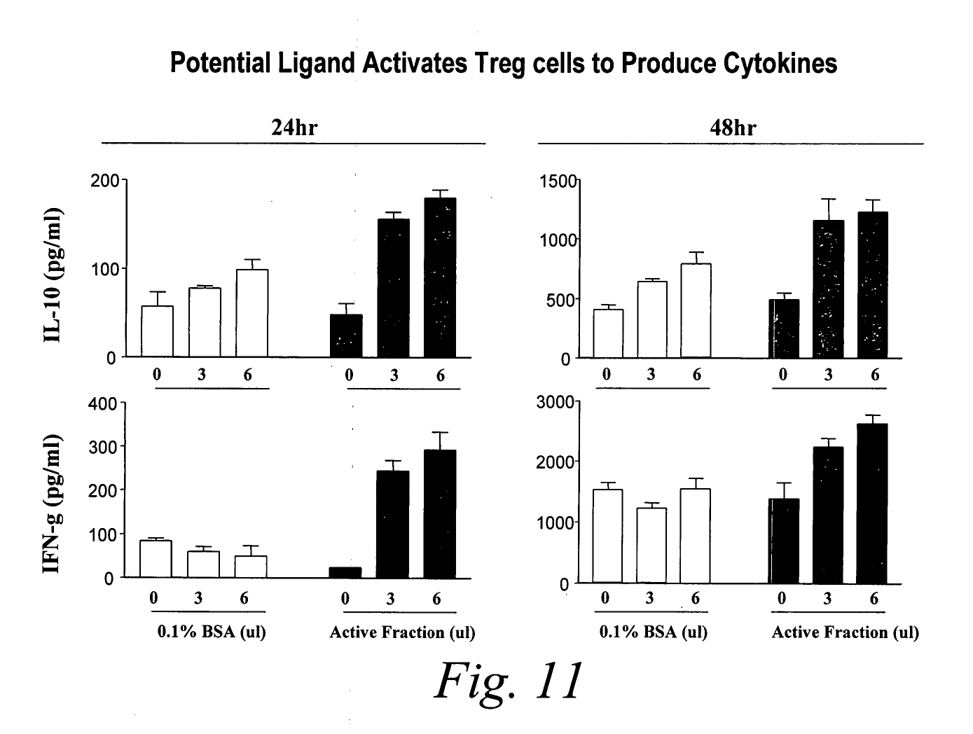

As indicated in FIG. 11, IL-10 and IFN-gamma (Treg derived cytokines known to be key players in immunoregulation) were profoundly increased in the culture containing the mouse brain derived active fraction (containing a ligand for GPR83) in a dose-dependent manner. The data in FIGS. 10 and 11 clearly demonstrate that the GPR83 ligand contained in the mouse brain derived fraction specifically stimulates Treg cells and augments their immunoregulatory/immunoinhibitory activities.

Because the responder CD25−CD4+ T cells contain autoreactive pathogenic T cells which can induce fatal autoimmune diseases in, for example, the immunodeficiency SCID or Nude mice, the GPR83 agonist contained within the murine brain derived fraction may be used to control, treat or prevent autoimmune diseases. In addition, exogenously transferred Treg cells can prevent the development of allergic reaction and rejection of transplantation graft in several preclinical mice models. Thus, the GPR83 agonist contained within the murine brain derived fraction may also be used to control, treat or prevent allergic diseases or graft rejection.

### Example 12

### Mouse Brain Derived Ligand for GPR83 Activates the Immunoregulatory Function of CD25⁺CD4⁺ Treg Cells

Aim of the Experiment

In order to confirm the results shown in FIGS. 10 and 11, an in vitro Treg assay was performed using a more physiologic stimulation which is initiated by a known antigen that is recognized by the antigen-specific T cells. In this assay, ovalubunime (OVA) peptide was used as the antigen to stimulate both T and Treg cells harboring an OVA-specific T cell receptor (TCR). T cells were prepared from OVA-specific TCR transgenic mice (DO 11.10 mice).

Materials and Methods

CD25⁻CD4⁺ T cells (5×10⁴cells/well) were purified from DO 11.10 mice, then cultured alone or with the indicated numbers of CD25⁺CD4⁺ T cells (2.5 or 5×10⁴/well) isolated from DO 11.10 in the presence of antigen presenting cells (APCs) (1×105/well) and OVA peptide (100 ng/ml) for 72 hr. Each well was pulsed with 500 nCi of [3H]thymidine for the last 6 h. The cells were then harvested on fiberglass filters and the incorporation of thymidine was measured with a beta-plate counter.

Results

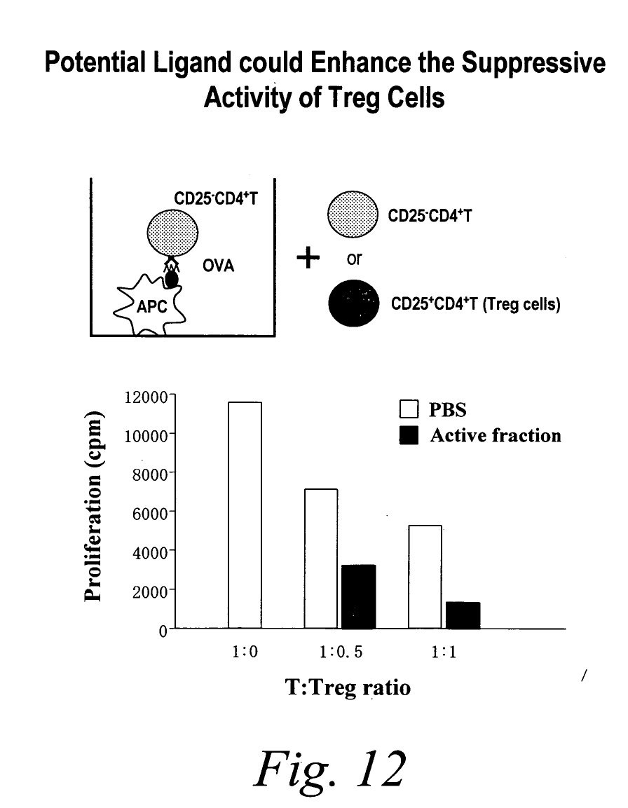

As indicated in FIG. 12, the anti-proliferative activity of CD25⁺CD4⁺ Treg cells against CD25⁻CD4⁺ T cells was clearly enhanced in the presence of the mouse brain derived active fraction (containing a GPR83 ligand).

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. An assay for identifying a GPR83 agonist capable of stimulating regulatory T cell function comprising:
contacting a test compound with an indicator composition comprising a GPR83 polypeptide; and
determining the ability of the test compound to stimulate the activity of the GPR83 polypeptide, wherein stimulation of the activity of the GPR83 polypeptide indicates that the test compound is capable of stimulating a regulatory T cell function, thereby identifying the test compound as a GPR83 agonist capable of stimulating a regulatory T cell function.

2. An assay for identifying a GPR83 agonist capable of stimulating regulatory T cell function comprising:
contacting a test compound with an indicator composition comprising a GPR83 polypeptide; and
determining the ability of the test compound to stimulate a regulatory T cell function which is mediated by a GPR83 polypeptide, thereby identifying the test compound as a GPR83 agonist capable of stimulating a regulatory T cell function.

3. The method of claim 1, wherein the test compound is a member of a library of test compounds and wherein the indicator composition comprising a GPR83 polypeptide is contacted with each member of the library of test compounds.

4. The method of claim 1, wherein the test compound is a member of a library of test compounds and wherein the indicator composition comprising a GPR83 polypeptide is contacted with at least half the members of the library of test compounds.

5. The method of claim 1, further comprising determining the effect of the test compound on a T regulatory cell function using an in vivo assay.

6. The method of claim 5, wherein said in vivo assay comprises the use of an animal model for an allergic disease or an autoimmune disease.

7. The method of claim 1, wherein the indicator composition is a cell expressing a recombinant GPR83 polypeptide.

8. The method of claim 7, wherein the cell has been engineered to express the GPR83 polypeptide by introducing into the cell an expression vector encoding the GPR83 polypeptide.

9. The method of claim 1, wherein the method comprises measuring intracellular adenylyl cyclase activity or intracellular calcium concentration in the presence and in the absence of the test compound and subsequently testing the ability of the test compound to stimulate a regulatory T cell function.

10. The method of claim 1, wherein the indicator composition comprises an indicator cell, wherein the indicator cell comprises the GPR83 polypeptide and a reporter gene sensitive to an activity of the GPR83 polypeptide.

11. The method of claim 1, wherein the indicator composition is a Foxp3 containing T cell.

12. The method of claim 1, wherein the regulatory T cell function which is mediated by a GPR83 polypeptide is suppression of the production of an effector cytokine.

13. The method of claim 12, wherein said effector cytokine is IL-2.

14. The method of claim 1, wherein the regulatory T cell function which is mediated by a GPR83 polypeptide is suppression of the function of an effector T cell.

15. The method of claim 14, wherein said effector cell is selected from the group consisting of T helper cells and cytotoxic T cells.

16. The method of claim 15, wherein said T helper cells are selected from the group consisting of Th1 and Th2 cells.

17. The method of claim 1, wherein the regulatory T cell function which is mediated by a GPR83 polypeptide is suppression of the proliferation of Th1 or Th2 cells.

18. The method of claim 1, wherein the regulatory T cell function which is mediated by a GPR83 polypeptide is suppression of cytokine production by Th1 or Th2 cells.

19. An assay for identifying a GPR83 antagonist capable of suppressing regulatory T cell function comprising:
contacting a test compound with an indicator composition comprising a GPR83 polypeptide; and
determining the ability of the test compound to suppress a regulatory T cell function which is mediated by a GPR83 polypeptide, thereby identifying the test compound as a GPR83 antagonist capable of suppressing a regulatory T cell function.

20. The method of claim 19, wherein the test compound is a member of a library of test compounds and wherein the indicator composition comprising a GPR83 polypeptide is contacted with each member of the library of test compounds.

21. The method of claim 19, wherein the test compound is a member of a library of test compounds and wherein the indicator composition comprising a GPR83 polypeptide is contacted with at least half the members of the library of test compounds.

22. The method of claim 19, further comprising determining the effect of the test compound on a T regulatory cell function using an in vivo assay.

23. The method of claim 22, wherein said in vivo assay comprises the use of an animal model for HIV or an animal model of a tumor.

24. The method of claim 19, wherein the indicator composition is a cell expressing a recombinant GPR83 polypeptide.

25. The method of claim 24, wherein the cell has been engineered to express the GPR83 polypeptide by introducing into the cell an expression vector encoding the GPR83 polypeptide.

26. The method of claim 19, wherein the method comprises measuring intracellular adenylyl cyclase activity or intracellular calcium concentration in the presence and in the absence of the test compound and subsequently testing the ability of the test compound to suppress a regulatory T cell function.

27. The method of claim 19, wherein the indicator composition comprises an indicator cell, wherein the indicator cell comprises the GPR83 polypeptide and a reporter gene sensitive to an activity of the GPR83 polypeptide.

28. The method of claim 19, wherein the indicator composition is a Foxp3 containing T cell.

29. The method of claim 19, wherein the regulatory T cell function which is mediated by a GPR83 polypeptide is suppression of the production of an effector cytokine.

30. The method of claim 29, wherein said effector cytokine is IL-2.

31. The method of claim 19, wherein the regulatory T cell function which is mediated by a GPR83 polypeptide is suppression of the function of an effector T cell.

32. The method of claim 31, wherein said effector cell is selected from the group consisting of T helper cells and cytotoxic T cells.

33. The method of claim 32, wherein said T helper cells are selected from the group consisting of Th1 and Th2 cells.

34. The method of claim 19, wherein the regulatory T cell function which is mediated by a GPR83 polypeptide is suppression of the proliferation of Th1 or Th2 cells.

35. The method of claim 19, wherein the regulatory T cell function which is mediated by a GPR83 polypeptide is suppression of cytokine production by Th1 or Th2 cells.


SEQUENCE LISTING (10 sequences)

SEQ ID NO: 1 - DNA, 33 bp, Artificial Sequence
  Description of Artificial Sequence Synthetic Primer
cggaattccgcaccatgcccaaccctaggccag

SEQ ID NO: 2 - DNA, 32 bp, Artificial Sequence
  Description of Artificial Sequence Synthetic Primer
ccgctcgagcggtcaagggcagggattggagc

SEQ ID NO: 3 - DNA, 20 bp, Artificial Sequence
  Description of Artificial Sequence Synthetic Primer
caggccagactttgttggat

SEQ ID NO: 4 - DNA, 20 bp, Artificial Sequence
  Description of Artificial Sequence Synthetic Primer
ttgcgctcatcttaggcttt

SEQ ID NO: 5 - DNA, 20 bp, Artificial Sequence
  Description of Artificial Sequence Synthetic Primer
catctgggtcatggctacct

SEQ ID NO: 6 - DNA, 20 bp, Artificial Sequence
  Description of Artificial Sequence Synthetic Primer
gccaggtccagatacttcca

SEQ ID NO: 7 - DNA, 20 bp, Artificial Sequence
  Description of Artificial Sequence Synthetic Primer
ggagctggaaaaggagaagc

SEQ ID NO: 8 - DNA, 19 bp, Artificial Sequence
  Description of Artificial Sequence Synthetic Primer
gctacgatgcagcaagagc

SEQ ID NO: 9 - DNA, 35 bp, Artificial Sequence
  Description of Artificial Sequence Synthetic Primer
cgcgtcgacgccaccatgaaggttcctcctgtcct

SEQ ID NO: 10 - DNA, 32 bp, Artificial Sequence
  Description of Artificial Sequence Synthetic Primer
gcgggcggccgctttcaccgtggggttctggg